(19) United States
(12) Patent Application Publication
(10) Pub. No.: US 2022/0142907 A1
(43) Pub. Date: May 12, 2022

(54) MYCELIUM BIOPOLYMERS FOR HEALTH AND BEAUTY APPLICATIONS

(71) Applicant: ECOVATIVE DESIGN LLC, Green Island, NY (US)

(72) Inventors: Eben Bayer, Troy, NY (US); Gavin Reim McIntyre, Troy, NY (US); Alex James Carlton, Troy, NY (US); Meghan A. O'Brien, Halfmoon, NY (US); Peter Mueller, Poestenkill, NY (US); Damen Donald Schaak, Troy, NY (US); Jessie Hannah Kaplan-Bie, Troy, NY (US)

(21) Appl. No.: 17/435,575

(22) PCT Filed: Mar. 12, 2019
(86) PCT No.: PCT/US2020/022409

§ 371 Date: Sep. 1, 2021

(51) Int. Cl. A61K 8/9728; A61K 8/02; A61K 8/73; A61Q 19/00; A61K 8/65; C12N 1/14; A61K 9/70; A61K 47/46; A61K 47/36

(52) U.S. Cl. CPC A61K 8/9728; A61K 8/0216; A61K 8/736; A61Q 19/00; A61K 8/65; C12N 2533/90; A61K 8/73; C12N 1/145; A61K 9/7007; A61K 47/46; A61K 47/36; A61K 8/735

(57) ABSTRACT

Topical applicators containing mycological biopolymers are suitable for applying health or cosmetic products to the skin or lips of a subject. The applicators are made from biodegradable mycological biopolymers that are grown from fungi in the presence of a growth medium under a predetermined environment of relative humidity, temperature, carbon dioxide, oxygen and air flow. The topical applicators containing the mycological biopolymers may further contain beneficial agents or products that that may be transferred to the skin or lips, thereby enhancing the health or beauty of a subject.

### CROSS-REFERENCE TO RELATED APPLICATIONS

This application claims the benefit of U.S. Provisional Patent Application No. 62/817,599, filed Mar. 13, 2019.

### TECHNICAL FIELD

This disclosure relates to topical applicators containing mycological biopolymers. The topical applicators are suitable for use for health and beauty applications.

### BACKGROUND

The health and beauty industries leverage several different materials to serve as matrices or scaffolding to carry health or cosmetic ingredients or products to application regions, such as the skin or lips. Examples of materials that presently serve in these functions are detailed below.

| | Materials of | |
|---|---|---|
| Applicator | Construction | Applicator Characteristics |
| Cosmetics | Polyurethane, | These closed cell foams do not sorb |
| applicator | Silicone | the product and permit the user to |
| or sponge | | easily apply the product to the |
| | | application region. Serving in both |
| | | durable (reusable) and nondurable |
| | | (single use) applications. These foams |
| | | can be injection molded to various |
| | | densities and hardness to achieve |
| | | complex, three dimensional features, |
| | | or can be die cut from a sheet. |
| Facial | Cellulose, | These open cell foams sorb product |
| cleansing | polyurethane, | for removal from a former application |
| sponge | natural sponge | region. These materials are |
| | | predominately disposable after an |
| | | initial use. |
| Toe and/or | Ethylene vinyl | These products are |
| finger | acetate, poly | predominately closed cell foams that |
| spacer | vinyl chloride, | provide support, cushioning and |
| | polyurethane | resilience while separating toes or |
| | | fingers that have had their nails |
| | | painted. These products tend to be die |
| | | cut from a sheet. |
| Facial, hand | Polyester, | These are nonwoven and open cell |
| and foot | cellulose | foam products that are infused with an |
| masks | | agent or formulated product. These |
| | | are single-use, disposable products |
| | | that are typically die cut from a larger |
| | | sheet of material. |

As is known, various techniques have been used to grow a mycological biopolymer material without the production of a fruiting body, such as a stipe, pileus, gill or pore structure. The term mycological biopolymer as disclosed herein is interchangeable with “mycelial mat” or mycelial tissue.”

U.S. Published Patent Application 2015/0033620, the entire content of which is hereby incorporated by reference in its entirety, describes techniques for growing a material comprising fungal mycelium, referred to as “mycological biopolymer.” As described therein, a mycological biopolymer product provided by the disclosed method is characterized as containing a homogenous biopolymer matrix that is comprised predominantly of fungal chitin and trace residues (e.g., beta-glucan, proteins). The mycological biopolymer is up-cycled from domestic agricultural lignocellulosic waste and is made by inoculating the domestic agricultural lignocellulosic waste substrate with a selected fungus in a container that is sealed off from the ambient environment external to the container. In addition to the substrate and fungal inoculum, the container contains a void space, which space is subsequently filled with a network of undifferentiated fungal mycelium. The biopolymer product grows into the void space of the container, filling the space with an undifferentiated mycelium comprising a chitin-polymer. The chitin-polymer-based mycelium is subsequently extracted from the substrate and dried. As further described in 2015/0033620, the environmental conditions for producing the mycological biopolymer product described therein, i.e. a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

As described in WO2019/099474, the entire content of which is hereby incorporated by reference in its entirety, another method of growing a biopolymer material employs incubation of a growth media comprised of a nutritive substrate inoculated with a fungus in containers that are placed in a closed incubation chamber with air flows passed over each container while the chamber is maintained with a predetermined environment characterized by parameters including relative humidity, temperature, carbon dioxide (CO₂) level and oxygen (O₂) level. The inoculated growth media in each container is incubated for a period of time sufficient for the fungus to digest the nutritive substrate and produce a mycelium biopolymer consisting entirely of fungal mycelium in each container.

As described in U.S. Ser. No. 16/519,384, filed Jul. 23, 2019, the entire content of which is hereby incorporated by reference in its entirety, a panel of biopolymer material as described in WO2019/099474 may be modified to generate a material with a custom texture, flavor, and nutritional profile for use as a foodstuff or a tissue scaffold. The method involves tailoring the density, morphology, and composition of the undifferentiated fungal material during growth and/or the use of post-processes, to improve mouth-feel and/or affinity toward flavors, fats, cellular cultures, or the like.

As described in U.S. Ser. No. 18/773,272, filed Jan. 27, 2020, the entire contents of which is hereby incorporated by reference in its entirety, another method of growing a biopolymer material employs a growth medium other than domestic agricultural lignocellulosic waste, particularly, alternatives to corn stover, that will reliably elicit mycological biopolymer growth without inducing the formation of visible fungal fruiting bodies, such as a stipe, pileus, gill or pore structure.

As described in WO 2019/099474 A1, the entire contents of which is hereby incorporated by reference in its entirety, another method of growing a biopolymer material employs incubation of a growth media comprised of nutritive substrate innoculated with a fungus in containers that are placed in a closed incubation chamber with air flows passed over each container while the chamber is maintained with predetermined environment of humidity, temperature, carbon dioxide and oxygen.

It is an object of the invention to provide a topical applicator containing a mycological biopolymer that is biodegradable and suitable for application of a health and/or beauty product.

It is another object of the invention to provide a method of making a topical applicator containing a mycological biopolymer that is biodegradable and suitable for the topical application of a health or beauty product.

It is yet another object of the invention to provide a method of enhancing the health or beauty of a subject.

### SUMMARY

Briefly, the present disclosure provides a topical applicator comprising a mycological biopolymer that can be grown and processed for many uses, including applications presently and predominately served by synthetic open and closed cell plastic foams and nonwoven synthetic materials.

As disclosed herein, the mycological biopolymer provides the topical applicator with a structural matrix and can further provide a source of nutrients or other beneficial ingredients that are optionally transferrable from the matrix to an application region of a subject, such as the skin or lips. Desirable physico-chemical properties of the mycological biopolymer are tuned during its growth cycle and/or down-stream processing, rendering it well suited for use as a topical applicator. For example, physical properties such as porosity (open volume), density, fluid retention capacity and the dimensions of it hyphal filaments, together with its chemical composition, including its protein and sugar (e.g., polysaccharide or oligosaccharide) content, which can further contribute to its hydrophobicity profile, render the mycological biopolymer suitable for the uptake and subsequent delivery of a wide variety of health and beauty products to a person's skin or lips. The mycological biopolymer-based topical applicators of the present disclosure are beneficially soft to the touch, even when applied to or brushed across the skin. As a further benefit, the topical applicators can be infused with a wide variety of agents or products during or after manufacture for subsequent delivery to the skin or lips.

Unlike the topical applicators in current use, which are based on synthetic polymers that consume significant fossil fuels during their production and subsequently add to landfill waste upon their disposal, the topical applicators of the present disclosure are “grown” from natural-occurring organisms and materials and are thus biodegradable, rendering them advantageously environmentally friendly.

There remains a need for improved, environmentally friendly topical applicators to serve the health and beauty industries.

In a general aspect, the present disclosure provides topical applicator comprising a structural matrix, wherein the structural matrix comprises a mycological biopolymer. The topical applicator is suitable for use in applying a health or beauty product to an application region of a subject. The application region is the subject's skin or lips.

In some further aspects, the topical applicator is for use in applying a health or beauty product to an application region of a subject.

The topical applicator contains a mycological biopolymer produced by a method comprising:
providing a growth medium comprising a fungal inoculum and a substrate, said substrate optionally further comprising a supplemental source of nutrition, and said fungal inoculum comprising a fungus; and
incubating the growth medium as a solid-state culture for a period of time in a growth environment, wherein the growth environment has a relative humidity, a temperature, carbon dioxide (CO₂) and oxygen (O₂) sufficient to support growth of the mycological biopolymer without the formation of a visible fruiting body; thereby providing the mycological biopolymer.

The growth environment carbon dioxide level can be within a range of about 3% (v/v) to about 7% (v/v). In some more particular embodiments, the growth environment carbon dioxide level is within the range of about 5% (v/v) to about 7% (v/v).

The growth environment relative humidity can be at least about 95%, such as about 99%.

The growth environment temperature can range from about 56° F. to about 100° F.

The growth environment temperature can range from about 85° F. to about 95° F.
The growth environment temperature can range from about 85° F. to about 90° F.
The growth environment can have a directed air flow. The directed air flow can be a horizontal directed air flow.

The fungus can be a species of the genus Flammulina Ganoderma, Inonotus, Lentinula, Morchella or Trametes.
The fungus can be a species of the genus Ganoderma.
The fungus can be Ganoderma lucidum.
The fungus can be a species of the genus Morchella.

The mycological biopolymer can have a density ranging from about 0.1 to about 5 pounds per cubic foot.

The mycological biopolymer can have an open volume ranging from about 70% to about 99%, about 75% to about 99%, about 80% to about 99%, or about 80% to about 92% (v/v).

In some embodiments, the mycological biopolymer can have a density of at least about 3 pounds per cubic foot. The growth environment to obtain the mycological biopolymer can be further characterized as having a horizontal directed air flow, the air flow having a rate ranging from about 250 feet per minute to about 500 feet per minute. More particularly, the air flow can range from about 275 feet per minute to about 500 feet per minute. The mycological biopolymer can have a tensile strength ranging from about 25 to about 35 psi. Optionally, the fungus is a species of the genus Ganoderma.

In other embodiments, the mycological biopolymer can have a density ranging from about 0.8 to about 3 pounds per cubic foot. The growth environment to obtain the mycological biopolymer can be further characterized as having a horizontal directed air flow, the air flow having a rate ranging from about 100 feet per minute to about 250 feet per minute, or about 110 to about 250 feet per minute. The mycological biopolymer has a tensile strength ranging from about 10 to about 20 psi. Optionally, the fungus is a species of the genus Ganoderma.

In other embodiments, the mycological biopolymer can have a density ranging from about 0.5 to about 2 pounds per cubic foot. The growth environment to obtain the mycological biopolymer can be further characterized as having a horizontal directed air flow, the air flow having an air flow rate of at most about 100 feet per minute, or less than about 100 feet per minute. Optionally, the fungus is a species of the genus Ganoderma. Optionally, the growth environment is a zero air flow environment.

In other embodiments, the mycological biopolymer can have a density of less than about 1 pound per cubic foot. Optionally, the fungus is a species of the genus Morchella.

The growth medium can be incubated in the growth environment as the solid-state culture for a period of time of up to about 3 weeks. The period of time can be from about 4 days to about 14 days. The period of time can be about 9 days.

The mycological biopolymer can have a fluid retention capacity of up to about 20-fold of the dry mass of the mycological biopolymer.

The mycological biopolymer can have hypha filaments having a thickness ranging from about 0.5 micron to about 10 microns, or from about 0.5 micron to about 6 microns.

The mycological biopolymer can have a modulus of elasticity ranging from about 1 to about 100 psi.

The mycological biopolymer can have a modulus of elasticity ranging from about 30 to about 60 psi, about 35 to about 80 psi, or about 35 to about 55 psi.

The mycological biopolymer can have a mat height ranging from about 0.125 inch to about 3 inches.

The mycological biopolymer can have a hydrophobicity characterized by a contact angle ranging from about 90 to about 150 degrees, about 100 to about 130 degrees, or about 110 to about 125 degrees.

The mycological biopolymer can have a chitin content ranging from about 5% to about 35%, about 5% to about 25%, or about 5% to about 15%, about 8 to about 12%, or about 10% (w/w) of the dry mass of the mycological biopolymer.

The mycological biopolymer can have a protein content ranging from about 1% to about 25%, about 2% to about 20%, about 5% to about 10%, or about 6% to about 9% (w/w) of the dry mass of the mycological biopolymer.

The topical applicator structural matrix can consist essentially of the mycological biopolymer. The topical applicator structural matrix can consist of the mycological biopolymer.

The topical applicator can consist of the mycological biopolymer.

Alternatively, the topical applicator can contain an additional substance (beyond the mycological biopolymer). The additional substance is a health or beauty product. The health or beauty product can be suitable for topical administration.

The additional substance can be an anesthetic, an analgesic, an antimicrobial, an antibiotic, an antiseptic, an anti-inflammatory, an exfoliant, a cosmetic, a sunscreen, a sun lotion, a moisturizer, a topical burn treatment agent, a cleanser, an astringent, a toner, a chelator, an anti-aging product, an anti-acne agent, an anti-coagulant, a protein, a signaling molecule, a complex carbohydrate, a pigment, a vitamin, a nutritional supplement, an oil or a microbe; or a combination thereof.

The additional substance can be a protein. The protein can be collagen.

The additional substance can be a complex carbohydrate. The complex carbohydrate can be hyaluronic acid, beta-glucan, or a combination thereof. The beta-glucan can include water soluble beta-glucan.

The additional substance can be added to the mycological biopolymer after the growth of the mycological biopolymer is terminated.

The additional substance can be a microbe. The microbe can be added during the growth of the mycological biopolymer.

The topical applicator can be in the form of a sheet, a circular disc, a triangle, a cylinder, a rectangle or a cone, or any other 3-dimensional molded form or custom design.

The topical applicator can be a mask, a finger spacer, a toe spacer, a cleansing foam, a wipe, an ear plug, a product applicator or a product blender. The product applicator can be a cosmetics applicator. The product blender can be a cosmetics blender.

The topical applicator can be for use in the application of a beauty product. The beauty product can be a cosmetic.

The topical applicator can be for use in the application of a health product. The topical applicator can be a bandage, a burn dressing or a wound dressing.

The mycological biopolymer is biodegradable.

The mycological biopolymer can be grown using a growth medium substrate that is a lignocellulosic substrate.

The lignocellulosic substrate can be an agricultural waste product. The agriculture waste product can be corn stover, kenaf pith, canola straw or wheat straw. Alternatively, the lignocellulosic substrate can exclude an agricultural waste product. The non-agricultural waste lignocellulosic substrate can be a plant or tree flour. The tree flour can be maple wood flour. The plant flour can be soy flour.

The mycological biopolymer can be grown using a growth medium substrate that is a cellulosic substrate. The cellulosic substrate can be a lignin-free material.

The mycological biopolymer can be grown using a growth medium substrate that is an inorganic substrate. The inorganic substrate can be vermiculite, perlite, soils, chalk, gypsum, clay, send, rockwool, expanded clay or growstones.

The fungus can be genetically engineered.

The fungus can be genetically engineered to overexpress a chitin deacetylase (DCA) gene.

The fungus can be genetically engineered to overexpress hydrophobins.

The fungus can be of the genus Ganoderma genetically engineered to overexpress the genes BGS1 and BGS2 that encode the two β-1,3-glucan synthases therein.

In another general aspect, there is provided a method of manufacturing a topical applicator of the present disclosure, which contains a mycological biopolymer, as disclosed herein. The method includes:
providing a growth medium comprising a fungal inoculum and a substrate, said substrate optionally further comprising a supplemental source of nutrition, and said fungal inoculum comprising a fungus; and
incubating the growth medium as a solid-state culture for a period of time in a growth environment, wherein the growth environment has a relative humidity, a temperature, carbon dioxide (CO₂) and oxygen (O₂) sufficient to support growth of the mycological biopolymer without the formation of a visible fruiting body; thereby providing the mycological biopolymer.

The growth environment carbon dioxide level can be within a range of about 3% (v/v) to about 7% (v/v). In some more particular embodiments, the growth environment carbon dioxide level is within the range of about 5% (v/v) to about 7% (v/v).

The growth environment relative humidity can be at least about 95%, such as about 99%.

The growth environment temperature can range from about 55° F. to about 100° F.

The growth environment temperature can range from about 85° F. to about 95° F.
The growth environment temperature can range from about 85° F. to about 90° F.
The growth environment can have a directed air flow. The directed air flow can be a horizontal directed air flow.

The growth medium can be incubated in the growth environment as the solid-state culture for a period of time of up to about 3 weeks. The period of time can be from about 4 days to about 14 days. The period of time can be about 9 days.

The fungus can be a species of the genus Flammulina Ganoderma, Inonotus, Lentinula, Morchella or Trametes.
The fungus can be a species of the genus Ganoderma.
The fungus can be Ganoderma lucidum.
The fungus can be a species of the genus Morchella.

The fungus can be genetically engineered.

The fungus can be genetically engineered to overexpress a chitin deacetylase (DCA) gene.

The fungus can be genetically engineered to overexpress hydrophobins.

The fungus can be of the genus Ganoderma genetically engineered to overexpress the genes BGS1 and BGS2 that encode the two β-1,3-glucan synthases therein.

The growth environment to obtain the mycological biopolymer can be further characterized as having a horizontal directed air flow, the air flow having a rate ranging from about 250 feet per minute to about 500 feet per minute. More particularly, the air flow can range from about 275 feet per minute to about 500 feet per minute.

Alternatively, the growth environment to obtain the mycological biopolymer can be further characterized as having a horizontal directed air flow, the air flow having a rate ranging from about 100 feet per minute to about 250 feet per minute, or about 110 to about 250 feet per minute.

Alternatively, the growth environment to obtain the mycological biopolymer can be further characterized as having a horizontal directed air flow, the air flow having an air flow rate of at most about 100 feet per minute, or less than about 100 feet per minute. The growth environment to obtain the mycological biopolymer can be further characterized as having a horizontal directed air flow, the air flow having an air flow rate of at most about 100 feet per minute, or less than about 100 feet per minute.

The mycological biopolymer can be grown using a growth medium substrate that is a lignocellulosic substrate.

The lignocellulosic substrate can be an agricultural waste product. The agriculture waste product can be corn stover, kenaf pith, canola straw or wheat straw. Alternatively, the lignocellulosic substrate can exclude an agricultural waste product. The non-agricultural waste lignocellulosic substrate can be a plant or tree flour. The tree flour can be maple wood flour. The plant flour can be soy flour.

The mycological biopolymer can be grown using a growth medium substrate that is a cellulosic substrate. The cellulosic substrate can be a lignin-free material.

The mycological biopolymer can be grown using a growth medium substrate that is an inorganic substrate. The inorganic substrate can be vermiculite, perlite, soils, chalk, gypsum, clay, sand, rockwool, expanded clay or growstones.

The method can further comprise incorporated an additional substance into the mycological biopolymer, or the topical applicator prepared from the mycological biopolymer. The additional substance can be an anesthetic, an analgesic, an antimicrobial, an antibiotic, an antiseptic, an anti-inflammatory, an exfoliant, a cosmetic, a sunscreen, a sun lotion, a moisturizer, a topical burn treatment agent, a cleanser, an astringent, a toner, a chelator, an anti-aging product, an anti-acne agent, an anti-coagulant, a protein, a signaling molecule, a complex carbohydrate, a pigment, a vitamin, a nutritional supplement, an oil or a microbe; or a combination thereof.

The method can comprise incorporating a protein into the mycological biopolymer or the topical applicator comprising the mycological biopolymer. The protein can be collagen.

The method can comprise incorporating a complex carbohydrate into the mycological biopolymer or the topical applicator comprising the mycological biopolymer. The complex carbohydrate can be hyaluronic acid, beta-glucan, or a combination thereof. The beta-glucan can include water soluble beta-glucan.

The method can comprise adding the additional substance to the mycological biopolymer after the growth of the mycological biopolymer is terminated.

The method can comprise incorporating a microbe into the mycological biopolymer or the topical applicator comprising the mycological biopolymer. The microbe can be added during the growth of the mycological biopolymer.

The method further comprises providing the topical applicator in a form suitable for use in applying a heath or beauty product to a subject. The form can be a sheet, a circular disc, a triangle, a cylinder, a rectangle or a cone, or any other 3-dimensional molded form or custom design. The form can be a mask, a finger spacer, a toe spacer, a cleansing foam, a wipe, an ear plug, a product applicator or a product blender.

In another general aspect, there is provided a method of enhancing the health a subject, the method comprising using a topical applicator as disclosed herein to apply a health product to an application region of a subject in need thereof.

In another general aspect, there is provided a method of enhancing the aesthetic appearance of a subject, the method comprising using a topical applicator as disclosed herein to apply a beauty product to an application region of a subject in need thereof.

These and other objects and advantages will become more apparent from the following detailed description taken with the accompanying drawings.

### BRIEF DESCRIPTION OF THE DRAWINGS

FIG. 1 shows a photograph of the top surface of a mycological biopolymer panel grown from Ganoderma sp. in a direct, high airflow environment; the panel exhibits minimal heterogeneity in mycelial tissue morphology.

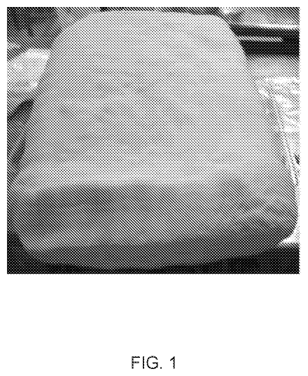

FIG. 2 shows a photograph of the top surface of a mycological biopolymer panel grown from Ganoderma sp. in an indirect, low airflow environment; the panel exhibits a high degree of heterogeneity in mycelial tissue.

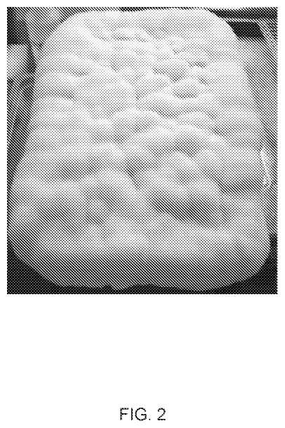

FIG. 3 shows a photograph of the top surface of a mycological biopolymer panel grown from Ganoderma sp. in a zero-airflow environment; the panel exhibits a high degree of heterogeneity in the mycelial tissue and reduced aerial growth.

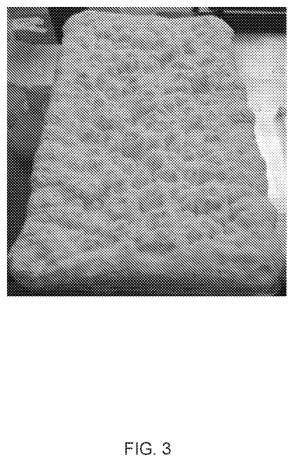

FIG. 4 shows micron scale hyphal filaments of a mycological biopolymer grown from Ganoderma sp. incubated with a direct horizontal airflow in the range of 100 to 250 feet/meter.

FIG. 5 shows a photograph of mycological biopolymer grown from Morchella sp.

### DETAILED DESCRIPTION

The present disclosure is directed to mycological biopolymer compositions, and more particularly, mycological biopolymers suitable for use as topical applicators. Also disclosed are methods of preparing the topical applicators, and methods of using the applicators for applying health or beauty products to the skin or lips, thereby enhancing the health and/or beauty of a subject.

The following disclosure illustrates aspects of the compositions, uses thereof, and methods of making the compositions embodied in the claims.

### Topical Applicators Containing Mycological Biopolymers

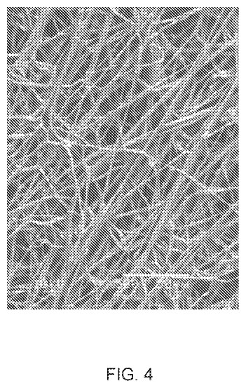

A mycological biopolymer of the present disclosure is an aerial matrix characterized as having cylindrical, micron scale hyphal filaments that collectively resemble an open-celled foam but with much finer structural resolution (FIG. 4). These mycological biopolymers are suitable for use as topical applicators to carry and apply, or even to remove, health and/or beauty products to an area or region of a subject, sometimes referred to as an application region. An application region may be, for example, the skin or lips of a subject.

The topical applicators of the present disclosure comprise mycological biopolymers characterized by one or more features, including but not limited to open volume (sometimes referred to as porosity), hyphal filament diameter and/or radius, fluid retention capacity, density, tensile strength, elastic modulus, hydrophobicity or hydrophilicity, durability and mat height. The mycological biopolymers are further characterized by their chemical composition, such as polysaccharide content (e.g., chitin content and/or beta-glucan content), protein content (e.g., hydrophobin content), or both; and optionally, pigment content.

The topical applicator of the present disclosure comprises a mycological biopolymer that is characterized as having an open volume. Accordingly, in some embodiments, the mycological biopolymer, or the topical applicator comprising the mycological biopolymer, has a mean open volume ranging from about 50% to about 99%, about 60% to about 99%, about 70% to about 99%, about 75% to about 99%, about 75% to about 95% (v/v); preferably, about 80% to about 99% (v/v). In some embodiments, the mycological biopolymer, or the topical applicator comprising the mycological biopolymer, has an open volume ranging from about 80% to about 92% (v/v).

In some embodiments, the topical applicator comprises a mycological biopolymer having a hyphal filament thickness ranging from about 0.5 micron to about 10 microns, or more particularly, from about 0.5 micron to about 6 microns.

In some embodiments, the mycological biopolymer, or the topical applicator comprising the mycological biopolymer, is characterized by its density, or more particularly, by the density of its dry mass. As used herein, “dry mass” of the mycological biopolymer may include about 8% to 10% residual moisture. In some embodiments, the density of the dry mass of the mycological biopolymer (or the topical applicator comprising the mycological biopolymer) ranges from about 0.1 to about 5 pounds per cubic foot. The dry mass is determined by methods known by a person skilled in the art after the mycological biopolymer (or the topical applicator) has undergone a drying process, such as but not limited to convection drying, ambient air drying or lyophilizing. In some embodiments, the mycological biopolymer is dried until it is found to contain about 10% or less residual moisture.

In some more particular embodiments, the mycological biopolymer has a relatively high density of greater than about 3 pounds per cubic foot. In some further embodiments, the density is up to about 5 pounds per cubic foot. In some embodiments, the higher density mycological biopolymer is produced in a growth environment having a horizontal directed air flow having an air flow rate in the range of about 250 feet per minute to about 500 feet per minute. In some even more particular embodiments, the directed air flow is a horizontal airflow has an air flow rate of greater than 250 feet per minute and up to about 500 feet, or about 275 feet per minute to about 500 feet per minute. In some further embodiments, the mycological biopolymer is produced by a species of the genus Ganoderma; optionally, the fungus is Ganoderma lucidum. In some further embodiments, the mycological biopolymer has a tensile strength of about 25 to about 35 psi.

In some other more particular embodiments, the mycological biopolymer has a relatively moderate density ranging from about 0.8 to about 3 pounds per cubic foot. In some embodiments, the moderate density mycological biopolymer is produced in a growth environment having a horizontal directed air flow, wherein the air flow has a rate in the range of about 100 feet per minute to about 250 feet per minute. In some further embodiments, the air flow rate is greater than about 100 feet per minute, for example, at least about 110 feet per minute, and up to about 250 feet per minute. In yet further embodiments, the mycological biopolymer is produced by a species of the genus Ganoderma; optionally, the fungus is Ganoderma lucidum. In some further embodiments, the mycological biopolymer has a tensile strength of about 10 to about 20 psi.

In yet other more particular embodiments, the mycological biopolymer has a relatively low density ranging from about 0.5 to about 2 pounds per cubic foot. In some even more particular embodiments, the density of the dry mass of the mycological biopolymer (or the topical applicator) is about 0.5, about 0.6, about 0.7, about 0.8, about 0.9, about 1.0, about 1.1, about 1.2, about 1.3, about 1.4 or about 1.5 pound per cubic foot. In some embodiments, the lower density mycological biopolymer is produced in a growth environment having a horizontal directed air flow at a rate of at most about 100 feet per minute, or less than about 100 feet per minute. Alternatively, the lower density mycological biopolymer is produced in a growth environment is a zero air flow environment. In some further embodiments, the mycological biopolymer is produced by a species of the genus Ganoderma; optionally, the fungus is Ganoderma lucidum.

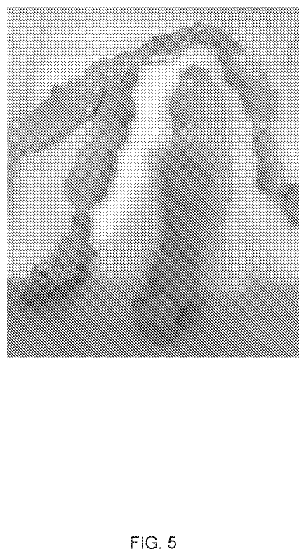

In yet some other more particular embodiments, the mycological biopolymer has an ultra-low density of less than about one pound per cubic foot. In a more particular embodiment, the mycological biopolymer is produced by a species of the genus Morchella [see FIG. 5].

In some embodiments, the mycological biopolymer, or the topical applicator comprising the mycological biopolymer, is characterized by its tensile strength. In some embodiments, the tensile strength ranges from about 10 psi to about 40 psi. In some embodiments, the tensile strength ranges from about 10 psi to about 20 psi, or about 12 psi to about 18 psi. In other embodiments, the tensile strength is greater than about 20 psi and upwards of about 40 psi, or from about 25 psi to about 36 psi.

In some embodiments, the mycological biopolymer, or the topical applicator comprising the mycological biopolymer, is characterized by a modulus of elasticity. In some embodiments, the mean modulus of elasticity of the mycological biopolymer, or the topical applicator comprising the mycological biopolymer, ranges from about 0.1 to about 120 psi, or more particularly, from about 1 to about 100 psi, about 5 to about 75 psi, about 10 to about 60 psi, about 20 to about 60 psi, about 25 to about 60 psi, about 30 to about 60 psi, about 35 to about 60 psi, or about 35 to about 55 psi.

In some embodiments, the mycological biopolymer suitable for use in the manufacture of a topical applicator of the present disclosure is characterized by the height of the mycelial mat. In some embodiments, the mat height is greater than about 0.125 inch, or at least about 0.25 inch. In some further embodiments, the mat height is at least about 0.5 inch, at least about 0.75 inch or at least about 1 inch. In some further embodiments, the mat height is at most about 4, about 5, or even about 6 inches. In some more particular embodiments, the mat height ranges from about 0.125 inch to about 3 inches.

In some embodiments, the topical applicator comprising the mycological biopolymer has a particular thickness, form or shape. In some embodiments, the topical applicator is provided as a sheet, a circular disc, a triangle, a cylinder, a rectangle, a cone, or any other 3-dimensional molded form or custom design. In some more particular embodiments, the topical applicator is provided as a mask (for example, a face mask, a hand mask or foot mask); a wipe (for example, a disinfectant wipe); a finger space or toe spacer; a skin cleansing foam; a bandage; a burn or wound dressing; or a cosmetic (or other product) applicator or remover (for example, for application or removal of a cosmetic to the skin or lips). In some embodiments, the topical applicator is characterized by its thickness. In some embodiments, the topical applicator thickness is the same or similar to the thickness of the mat of mycological biopolymer used in the manufacture of the topical applicator. Thus, in some embodiments, the topical applicator thickness is at least about 0.25 inch. In some further embodiments, the topical applicator thickness is at least about 0.5 inch, at least about 0.75 inch or at least about 1 inch. In some further embodiments, the topical applicator thickness is at most about 4, about 5 or about 6 inches. In some more particular embodiments, the topical applicator thickness ranges from about 0.125 inch to about 3 inches. In some alternative embodiments, the topical applicator is thinner in character. In some particular embodiments, the topical applicator thickness ranges from about 0.1 mm to about 5 mm, or about 0.5 to about 5 mm; for example, about 0.5 mm, about 1 mm, about 2 mm, about 3 mm, about 4 mm or about 5 mm thick. In some such embodiments, the topical applicator is a face mask or eye mask, or when the applicator is a wipe.

In some embodiments, the mycological biopolymer, or the topical applicator comprising the mycological biopolymer, is characterized by its sugar (e.g., polysaccharide or oligosaccharide content), for example, chitin content, beta-glucan content, or both. Chitin, a homopolymer found in the cell wall of the fungus, is the same substance commonly found in crustacean shells, and is both heat resistant (degradation at 280° C.) and water insoluble. Present estimates for the chitin composition of a Basidiomycete (e.g., a Ganoderma sp.) are between 10 and 15% of the mycelial tissue's dry mass. The chitinous cell wall is further encapsulated in a matrix of beta-glucans, which are composed of D-glucose units with beta-1-3 and beta-1-6-glucosidic bonds. Depending on the molecular weight of the beta-glucan polymer, this matrix is hydrophilic, with varying degrees of aqueous solubility. Furthermore, the beta-glucan matrix has been found to further polymerize under heat and pressure to preserve a desired structure or form as detailed in US2016/0302365, the entire content of which is hereby incorporated by reference in its entirety. The aforementioned cellular components are produced through a number of factors including substrate composition, environmental stimuli, and genomic transcription. As such, the composition of the fungal cell wall can be modulated through these factors to regulate the hydrophobicity, hydrophilicity, and porosity (open volume) of the mycological biopolymer.

Beta-glucans have been claimed in health and beauty products to hydrate skin, acting as a humectant and resolving redness common with skin conditions such as eczema or dermatitis. Oats, for example, are rich in beta-glucans, 15-35% of dry mass, and have been historically used in moisturizing soaps and other skincare products.

The beta-glucan content within the mycological biopolymer can provide the same dermatological value, while also serving as the matrix for product delivery. Further, the mycological biopolymer can be augmented, infused or imbibed with additional ingredients or be chemically functionalized to provide additional value (e.g., the deacetylation of chitin to create chitosan can offer inherent antimicrobial properties; creation of a hydrogel can further alter performance; see U.S. Pat. No. 9,555,395, the entire content of which is hereby incorporated by reference in its entirety.

In some embodiments, the mycological biopolymer, or the topical applicator comprising the mycological biopolymer, has a chitin content ranging from about 5% to about 35%, about 5% to about 25%, or about 5% to about 15%, about 8 to about 12%, or about 10% (w/w) of its dry mass. In some embodiments, the mycological biopolymer, or the topical applicator comprising the mycological biopolymer, has a beta-glucan content ranging from about 5% to about 35%, about 10% to about 35%, or about 15% to about 35% (w/w) of its dry mass. In some more particular embodiments, the mycological biopolymer, or the topical applicator comprising the mycological biopolymer, has a beta-glucan content ranging from about 10% to about 15%, for example, about 10%, about 11%, about 12%, about 13%, about 14% or about 15% (w/w) of its dry mass. The chitin and/or beta-glucan content of the mycological biopolymer, or the topical applicator comprising the mycological biopolymer, may be tuned to obtain a desired effect, as further disclosed herein. Methods of modulating chitin or beta-glucan content of a mycelium bound composite materials are disclosed, for example, in U.S. Ser. No. 16/363,052, published as U.S. 2019/0322997, the entire contents of which is hereby incorporated by reference in its entirety.

In some embodiments, the mycological biopolymer, or the topical applicator comprising the mycological biopolymer, is characterized by its aqueous solubility. In some embodiments, the aqueous solubility is imparted to the mycological biopolymer by its beta-glucan content, or more particularly, by the molecular weight or molecular weight distribution of the beta-glucans present in the mycological biopolymer.

In some embodiments, the mycological biopolymer, or the topical applicator comprising the mycological biopolymer, is characterized by its protein content. In some embodiments, the protein content ranges from about 1% to about 25%, about 2% to about 20%, about 5% to about 10%, or more particularly, about 6% to about 9% (w/w) of its dry mass. In some further embodiments, the mycological biopolymer, or the topical applicator comprising the mycological biopolymer, is characterized as having a protein content of about 5%, about 6%, about 7% about 8% about 9% or about 10% (w/w) of its dry mass. In some more particular embodiments, the mycological biopolymer, or the topical applicator comprising the mycological biopolymer, is characterized by its hydrophobin protein content. In some embodiments, the hydrophobin content is reported based on cysteine amino acid content of the mycological biopolymer. In some embodiments, the cysteine amino acid content ranges from about 0.05% to about 0.5%, or from about 0.1% to about 0.2% (w/w) of the mycological biopolymer. In some more particular embodiments, the cysteine amino acid content ranges from about 0.15% to about 0.17%, for example, about 0.165% (w/w) of the mycological biopolymer. In some embodiments, the hydrophobin content is indicated by a hydrophobic contact angle of the mycological biopolymer, as further disclosed herein. The hydrophobin content of the mycological biopolymer, or the topical applicator comprising the mycological biopolymer, may be tuned to obtain a desired effect, as further disclosed herein. Methods of modulating hydrophobin content of a mycelium bound composite material are disclosed, for example, in U.S. Ser. No. 16/363,052, published as U.S. 2019-0322997 A1, the entire contents of which is hereby incorporated by reference in its entirety.

In some embodiments, the mycological biopolymer, or the topical applicator comprising the mycological biopolymer, is characterized by its hydrophobicity. In some embodiments, the hydrophobicity is reported based on a contact angle. In some embodiments, the mycological biopolymer (or the topical applicator comprising the mycological biopolymer) has a contact angle ranging from about 90 to about 150 degrees, from about 100 to about 130 degrees, or more particularly, about 110 to about 125 degrees. In some embodiments, the hydrophobicity is imparted to the mycological biopolymer by the hydrophobin protein content of the biopolymer.

In some alternative embodiments, the mycological biopolymer, or the topical applicator comprising the mycological biopolymer, is characterized by its hydrophilicity, particularly when the hydrophobin content of the mycological biopolymer has been reduced to the point that the biopolymer is no longer hydrophobic in nature. In some embodiments, the hydrophilicity is reported based on a contact angle. In some embodiments, the mycological biopolymer (or the topical applicator comprising the mycological biopolymer) has a contact angle of greater than zero and less than about 90 degrees. In some more particular embodiments, the mycological biopolymer, or the topical applicator comprising the mycological biopolymer, has a contact angle of about 70 degrees to about 75 degrees. In some aspects, the present disclosure provides for methods of reducing the hydrophobicity of the mycological biopolymer. For example, a mycological biopolymer prepared according to methods of the present disclosure may be exposed to a polar protic solvent, such as an alcohol, to reduce the protein and/or beta-glucan content of the mycological biopolymer and providing a mycological biopolymer having a contact angle of less than about 90 degrees.

It is to be generally understood that the topical applicator of the present disclosure is suitable to carry and transfer (e.g., apply) a health or beauty product to a surface of the exterior of the body of a subject, for example, to the subject's skin or lips. Thus, in some embodiments, the topical applicator is characterized according to its fluid retention capacity).

In some embodiments, the mycological biopolymer, or the topical applicator comprising the mycological biopolymer, has a fluid retention capacity of up to about 20-fold of (20 times) the dry mass of the mycological biopolymer, or the topical applicator comprising the mycological biopolymer, respectively. In some further embodiments, the mycological biopolymer, or the topical applicator comprising the mycological biopolymer, has a fluid retention capacity of up to about 20-fold of the dry mass of the mycological biopolymer. In some embodiments, the fluid retention compacity ranges from about 0.1-fold to about 20-fold, about 0.5-fold to about 20-fold, about 1-fold to 20-fold, about 2-fold to about 20-fold, about 5-fold to about 20-fold, about 10-fold to about 20-fold, or about 10-fold to about 15-fold of the dry mass of the mycological biopolymer, or the topical applicator comprising the mycological biopolymer, respectively. In some embodiments, the fluid is a solution, such as an aqueous solution. In some embodiments, the fluid is an oil. In some embodiments, the fluid is formulated as a lotion, a gel, a suspension or an emulsion.

In some embodiments, the mycological biopolymer, or the topical applicator comprising the mycological biopolymer, is provided as a dry topical applicator that is not infused with an agent or formulated product during its manufacture. Regardless, the biopolymer or applicator, or the topical applicator comprising the mycological biopolymer, which can have a fluid retention capacity of up to 20-fold of (20 times) the dry mass of the mycological biopolymer, as disclosed herein, can be used to transfer an agent or formulated product to an application region. For example, the applicator can be infused with a fluid (for example, by a subject or consumer) just prior to use.

In some alternative embodiments, the mycological biopolymer, or the topical applicator comprising the mycological biopolymer, is a provided as a dry topical applicator which is not infused with an agent or formulated product during its manufacture, but is used to transfer an agent or formulated product to an application region at the time of use. In some such embodiments, the applicator preferably exhibits no or negligible retention of fluid or solid of a health or beauty product. Thus, in some aspects, the applicator comprising the mycological biopolymer and having no or negligible uptake or retention of the health or beauty product allows the product to be topically applied to the application region of the subject with no or negligible loss of the product to the applicator itself.

In some embodiments, the topical applicator of the present disclosure comprises a mycological biopolymer and one or more additional substances (as used herein, “additional substances” includes microorganisms). In some embodiments, the one or more additional substances is provided as a component of the topical applicator at the time of manufacture of the topical applicator. In other embodiments, the one or more additional substances is added to the topical applicator at the time of use of the topical applicator by a subject, for example, at the time when the subject prepares to apply the additional substance(s) to an application region.

In some embodiments, the additional substance is a health or beauty product, or more particularly, a topical healthcare or beauty product. A health or beauty product, as disclosed herein, include an agent or ingredient, or a formulated agent or ingredient. Nonlimiting examples of topical health or beauty products or ingredients include an anesthetic agent, an analgesic, an antimicrobial, an antibiotic, an antiseptic, an anti-inflammatory, an exfoliant, a cosmetic, a sunscreen, a sun lotion, a moisturizer (optionally in the form of microbeads), a topical burn treatment agent, a cleanser, an astringent, a toner, a chelator, an anti-aging product, an anti-acne agent, an anti-coagulant; a protein; a signaling molecule; a sugar (e.g., a polysaccharide or oligosaccharide); a pigment; a vitamin; a nutritional supplement; an oil; a microbe; or a combination thereof.

In some embodiments, the additional substance is an antimicrobial agent. Nonlimiting examples of an antimicrobial agent include silver chloride, silver sulfadiazine zinc chloride, iodine, cinnamaldehyde, a sorbate and tannins. In some further embodiments, the antimicrobial agent is silver chloride (AgCl) or zinc chloride (ZnCl), optionally present at a concentration of about 10 ppm to about 100 ppm of the mycological biopolymer.

In some embodiments, the additional substance is an anti-inflammatory agent or product. Nonlimiting examples of an anti-inflammatory agent or product include aloe vera, non-steroidal anti-inflammatory drugs (i.e., NSAIDS, such as ibuprofen, naproxen, diclofenac, celecoxib, aspirin), topical corticosteroids (e.g., triamcinolone, halobetasol, halcinonide, amcinonide, desoximetasone, desonide, mometasone, fluticasone, fluocinolone, flurandrenolide, fluticasone, diflorasone, fluocinonide, betamethasone, clobetasol, hydrocortisone, cortisone and prednicarbate; and pharmaceutically acceptable salts thereof); and combinations thereof.

In some embodiments, the additional substance is a topical burn treatment agent. Nonlimiting examples of topical agents for treating burns include antimicrobial agents (e.g., ointments), sliver-containing agents (e.g., silver sulfadiazine; Silvadene), bismuth-Impregnated petroleum chlorhexidine and mafenide.

In some embodiments, the additional substance is an analgesic agent. Nonlimiting examples of an analgesic agent include an NSAID (e.g., as disclosed herein), acetaminophen, an opioid (e.g., morphine) and cannabidiol.

In some embodiments, the additional substance is a protein. Nonlimiting examples of a protein include a collagen, a silk protein or a neurotoxic protein (e.g. Botulinum toxin or Botox®). In some embodiments, the protein is an enzyme. In some embodiments, the enzyme is capable of degrading a polysaccharide or oligosaccharide; nonlimiting examples of such enzymes include chitin deacetylase and hyaluronidase. In some embodiments, the enzyme is a proteolytic enzyme (i.e., a protease), optionally obtain from an extract of bacteria, fungi, animals or plants, such as fruits or vegetables. In some embodiments, the enzyme is bromelain (e.g., pineapple extract), papain (e.g., papaya extract), ficin (e.g., fig extract), or actinidin (e.g., kiwi, pineapple, mango, banana and/or papaya extract). In some embodiments, the enzyme provides the topical applicator with exfoliating properties. In some further embodiments, the enzyme is formulated at acidic pH (e.g., less than about pH 7, less than about pH 6.5, less than about pH 6, or less than about pH 5.5). In some embodiments, the enzyme is formulated as a cleanser.

In some embodiments, the additional substance is an exfoliant. In some embodiments, the exfoliant is an enzyme, as disclosed herein. In some other embodiments, the exfoliant is an alpha hydroxy acid, a beta hydroxy acid, or both. In some embodiments, the exfoliant is an enzyme as disclosed herein, an alpha hydroxy acid, a beta hydroxy acid; or a combination thereof. In yet another embodiment, the additional substance is an exfoliant and an anti-oxidant.

In some embodiments, the additional substance is a polysaccharide or oligosaccharide. Nonlimiting examples of a polysaccharide or oligosaccharide include chitin, hyaluronic acid and beta-glucan; and combinations thereof.

In some embodiments, the additional substance is an oil. Nonlimiting examples of an oil include coconut oil, olive oil (including extra virgin olive oil), avocado oil and jojoba oil; and combinations thereof. In some embodiments, the oil is an essential oil, such as lavender oil, Eucalyptus oil, chamomile oil, rose oil, hyssop oil, ylang ylang oil, myrrh oil, vetiver oil, frankincense oil, grapefruit oil, cedarwood oil, peppermint oil, spearmint oil, basil oil, Melaleuca (tea tree) oil, lemon oil, arborvitae oil, orange oil, Helichrysum oil, Cassia oil or oregano oil, and cannabidiol oil; and combinations thereof. In some embodiments, the oil is a combination of one or more essential oils and a carrier oil, such as coconut oil or jojoba oil.

In some embodiments, the additional substance is a toner. In some embodiments, a toner comprises one or more of the following ingredients including but not limited to aloe vera, willow bark, witch hazel, glycerin, salicylic acid, lactic acid, one or more essential oil (e.g., as disclosed herein), one or more vitamins (e.g., as disclosed herein), coenzyme Q10, hyaluronic acid; and combinations thereof.

In some embodiments, the additional substance is a vitamin. Nonlimiting examples of a vitamin include vitamin A, vitamin C, vitamin D, vitamin E, vitamin K, vitamin B1 (thiamine), vitamin B2 (riboflavin), vitamin B3 (niacin), pantothenic acid (B5), biotin (B7), vitamin B6, vitamin B12 (cyanocobalamin) and folate (folic acid and B9); and combinations thereof).

In some embodiments, the additional substance is an anesthetic agent, or more particularly, a topical anesthetic agent. Nonlimiting examples of topical anesthetic agents include benzocaine, butamben, dibucaine, lidocaine, oxybuprocaine, pramoxine, proparacaine, proxymetacaine and tetracaine (amethocaine).

In some embodiments, the additional substance is a microbe. In some embodiments, the microbe is a probiotic. In some embodiments, the probiotic comprises one or more species of Lactobacillus (e.g., L. rhamnosus, L. casei, L. paralisel, L. gasseri or L. salivarius; or a combination thereof). Bifidobacterium (e.g., B. infantis, B. bifidum, B. longum, B. breve or B. lactis; or a combination thereof); or a combination thereof.

In some embodiments, the additional substance is a pigment-producing microbe. Accordingly, in some embodiments, the mycological biopolymer, or the topical applicator comprising the mycological biopolymer, comprises an endogenous pigment produced by the fungus during the growth of the mycological biopolymer. That is to say, in some embodiments, the mycological biopolymer, or the topical applicator comprising the mycological biopolymer, is self-colorized. In some embodiments, the endogenous pigment is an anthraquinone, 1,1′-binaphthalene-4,4′-5,5′-tetrol (BNT), cinnabarinic acid, cytochalasin H, daldinal A, daldinal B, daldinal C, daldinal A, daldinal B, daldinal C, daldinal, daldiol, deoxyerythrostominone, deoxyerythrostominol, draconin red, epierythrostominol, erythrostominone, fragiformin A, fragiformin B, 2-hydroxy-5-methylchromone hypomiltin, hypoxylone, hypoxyxylerone, hypoxyvermethotin A, hypoxyvermelhotin B, hypoxyvermelhotin C, lenormandin A, lenormandin B, lenormandin C, lenormandin D, lenormandin E, lenormandin F, lenormandin G, melanin, 4-O-methyl erythrostominone 8-methoxy-1-napthol, mitorubrin, a mitorubrin azaphilone, mitorubrinal, naphthoquinone, rickenyl B, rickenyl D, rubiginosin, 3,5,8-trihydroxy-6-methoxy-2(5-oxohexa-1,3-dienyl)-1,4-naphthoquinone (3,5,8-TMON), vermelhotin, xylindein or xylindein quinol; or a derivative thereof; or a combination thereof. In alternative embodiments, any one of the foregoing pigments can be added to a mycological biopolymer during, or more preferably after the growth of the mycological biopolymer (so as not to be digested during the growth period), that is to say, the pigment present in the mycological biopolymer (or the topical applicator comprising the mycological biopolymer) is not required to be an endogenous pigment of the producing fungus. Thus, any pigment may be incorporated into the mycological biopolymer during, or preferably after its growth. In some alternative embodiments, the pigment is a botanical pigment. Nonlimiting examples of sources of botanical pigment include dried hibiscus, beet root, rose petals, and saffron.

The one or more additional substances provided as a component of the topical applicator (e.g., at the time of manufacture of the topical applicator, or added to the topical applicator at the time of use of the topical applicator by a subject may exist in a variety of physical forms. Thus, in some embodiments, the topical applicator comprises the mycological biopolymer and the one or more additional substance(s), wherein the one or more additional substance(s) is in the form of a liquid, a gel or a solid. In some embodiments, the one or more additional substance(s) is provided as a gel, an ointment, a lotion, a solution, a suspension, an emulsion, a cream, or a powder.

A mycological biopolymer of the present disclosure can be further characterized with reference to the organism from which it is grown. In some embodiments, the fungus is a species of the genus Amillaria, Agaricus, Agrocybe, Ceriporiopsis, Cerioporus, Chlorociboria, Cordyceps, Daldinia, Flammulina, Fomes, Ganoderma, Hericium Hypomyas, Hypoxylon, Hypsizygous, Inonotus, Laetiporus, Lentinula, Lentinus, Morchella, Omphalotus, Ophiocordyceps, Oxyporus, Phanerochaete, Picnoporus, Pleurotus, Polyporellus, Polyporus, Schizophyllum, Scytalidium, Trametes, Tuber or Xylaria.

In some embodiments, the fungus is a species of the genus Amillaria.

In some embodiments, the fungus is a species of the genus Agaricus.

In some embodiments, the fungus is a species of the genus Agrocybe.

In some embodiments, the fungus is a species of the genus Ceriporiopsis, for example, Cerioporus squamosus (formerly, Polyporus squamosus).

In some embodiments, the fungus is a species of the genus Chlorociboria, for example, Chlorociboria aeruginascens or Chlorociboria aeruginosa.

In some embodiments, the fungus is a species of the genus Cordyceps, for example, Cordyceps farinosa (formerly, Isaria farinosa).

In some embodiments, the fungus is a species of the genus Daldinia, for example, Daldinia bambusicol, Daldinia caldarium, Daldinia childiae, Daldinia clavate, Daltinia fissa, Daldinia grandis, Daldinia lloydi, Daldinia locuiata, Daldinia petriniae, Daldinia singularis, Daldinia concentrica or Daldinia eschscolzii.

In some embodiments, the fungus is a species of the genus Flammulina.

In some embodiments, the fungus is a species of the genus Fomes, for example, Fomes fomentarius.

In some embodiments, the fungus is a species of the genus Ganoderma. In some preferred embodiments, the fungus is Ganoderma tsugae, Ganoderma resinaceum or G. lucidum. In a more preferred embodiment, the fungus is Ganoderma lucidum.

In some embodiments, the fungus is a species of the genus Hericium.

In some embodiments, the fungus is a species of the genus Hypomyas, for example, Hypoxylon fragiformin, Hypoxylon howeanum, Hypoxylon lechatil, Hypoxylon fuscum, Hypoxylon fulvo-sulphureum, Hypoxylon sclerophaeum, Hypoxylon rickii, Hypoxylon lenormandli, Hypoxylon jaklitschii or Hypoxylon rubiginosin.

In some embodiments, the fungus is a species of the genus Hypsizygous.

In some embodiments, the fungus is a species of the genus Inonotus, for example, Inonotus hispidus.

In some embodiments, the fungus is a species of the genus Laetiporus.

In some embodiments, the fungus is a species of the genus Lentinula.

In some embodiments, the fungus is a species of the genus Lentinus, for example, Lentinus brumalis (formerly, Polyporus brumalis).

In some embodiments, the fungus is a species of the genus Morchella.

In some embodiments, the fungus is a species of the genus Omphalotus.

In some embodiments, the fungus is a species of the genus Ophiocordyceps, for example, Ophiocordyceps unilateralis (formerly, Cordyceps unilateralis).

In some embodiments, the fungus is a species of the genus Oxyporus, for example, Oxyporus populinus.

In some embodiments, the fungus is a species of the genus Phanerochaete.

In some embodiments, the fungus is a species of the genus Picnoporus, for example, Picnoporus cinnabarinus.

In some embodiments, the fungus is a species of the genus Pleurotus, for example, Pleurotus ostreatus.

In some embodiments, the fungus is a species of the genus Polyporellus.

In some embodiments, the fungus is a species of the genus Polyporus.

In some embodiments, the fungus is a species of the genus Schizophyllum.

In some embodiments, the fungus is a species of the genus Scytalidium, for example, Scytalidium cuboideum, Scytalidium, ganodermophthorum or Scytalidium lignocola.

In some embodiments, the fungus is a species of the genus Trametes, for example, Trametes versicolor.

In some embodiments, the fungus is a species of the genus Tuber, for example, Tuber melanosporum.

In some embodiments, the fungus is a species of the genus Xylaria, for example, Xylaria polymorpha.

### Process for Making Topical Applicators

In some aspects, the present disclosure provides methods of preparing a mycological biopolymer suitable for use as a topical applicator, as disclosed herein. The disclosed methods provide aerial mycological biopolymers that do not contain visible fruiting bodies, such as a stipe, pileus, gill or pore structure.

In some aspects, the method of preparing a mycological biopolymer suitable for use as a topical applicator comprises providing a growth media containing a substrate, optionally further containing a supplemental source of nutrition, and further containing a fungal inoculum, the fungal inoculum containing a fungus. Suitable fungi for preparing the mycological biopolymer are disclosed herein. In some further embodiments, the method comprises incubating the inoculated substrate and optional nutritional supplement as a solid-state culture for a period of time in a growth environment. In some embodiments, the growth environment is one characterized as having a relative humidity, a temperature, carbon dioxide (CO₂) and an oxygen (O₂) sufficient to support the growth of fungal mycelial tissue without the formation of a visible fruiting body, such as a stipe, pileus, gill or pore structure. Accordingly, in some embodiments, the fungal growth consists essentially of fungal mycelial tissue growth. Thus, in some embodiments, the mycological biopolymer so obtained consists essentially of the mycelial tissue. In some further embodiments, the fungal growth consists of fungal mycelial tissue growth. Thus, in some embodiments, the mycological biopolymer so obtained consists of the mycelial tissue.

In some embodiments, the growth environment is such that the carbon dioxide is present at a level ranging from about 3% (v/v) to about 7% (v/v). In some other embodiments, the carbon dioxide level ranges from about 5% (v/v) to about 7% (v/v).

In some embodiments, the growth environment is such that the temperature ranges from about 55° F. to about 100° F. In some embodiments, the growth environment temperature ranges from about 85° F. to about 95° F. In some more particular embodiments, the growth environment temperature ranges from about 85° F. to about 90° F.

In a further embodiment, the growth environment includes a directed air flow. In some embodiments, the air flow is directed parallel to the surface of the growth media (and to the resulting mycelial mat), i.e., the directed air flow is a horizontal air flow. In some embodiments, the air flow can be adjusted to modulate the homogeneity, density and/or tensile strength of the mycological biopolymer.

In some more particular embodiments, the directed air flow is a horizontal airflow having an air flow rate ranging from about 250 feet per minute to about 500 feet per minute. In some even more particular embodiments, the directed air flow is a horizontal airflow has an air flow rate of greater than 250 feet per minute and up to about 500 feet; or about 275 feet per minute to about 500 feet per minute.

In some other embodiments, the directed air flow is a horizontal airflow having an airflow rate ranging from about 100 feet per minute to about 250 feet per minute. In some further embodiments, the air flow rate is greater than about 100 feet per minute, for example, at least about 110 feet per minute, and up to about 250 feet per minute.

In some other embodiments, the directed air flow is a horizontal air flow having an air flow rate of at most about 100 feet per minute, or less than 100 feet per minute.

In some other embodiments, the growth environment is a zero-air flow environment.

In some embodiments, the growth environment of high relative humidity. In some embodiments, the relative humidity is at least about 95%. In some embodiments, the relative humidity is about 99%. In some other embodiments, the relative humidity is greater than 99%.

In some embodiments, the incubation time for the growth of the fungal mycelial tissue is up to about 3 weeks. In some other embodiments, the incubation time is up to about 2 weeks. In some more particular embodiments, the incubation time is about 4 days to about 14 days, for example, for about 9 days.

In some embodiments, the method of growing a mycological biopolymer of the present disclosure comprises providing a substrate to support growth of the mycological biopolymer. In some embodiments, the substrate is a natural substrate, a synthetic substrate or an artificial substrate, as disclosed herein. As used herein, “substrate”, refers to a medium that supports growth of a mycological biopolymer.

In some embodiments, the natural substrate for supporting growth of the mycological biopolymer comprises a lignocellulosic material. As used herein, “lignocellulosic material” refers to biomass comprising in pad cellulose and lignin. In some embodiments, the lignocellulosic material is, or is derived from, a plant or wood material.

In some embodiments, the lignocellulosic material is an agricultural waste product, such as corn stover, kenaf pith, canola straw or wheat straw.

In some alterative embodiments, the lignocellulosic material is not an agricultural waste product. For example, in some embodiments, the substrate is not corn stover. In some further embodiments, the plant or wood material is purposefully harvested for use in the production of the mycological biopolymer. Nonlimiting examples of suitable lignocellulosic substrate comprising or obtained from plant or wood material include hemp, maple, corn, kenaf, canola, soy, wheat, seed, seed husk, and the like, in some embodiments, seed(s), seed husks, or both include sunflower seed, walnut, poppy seed, etc. Nonlimiting examples of tree material include hardwoods and softwoods, including from the genus Acer, Quercus, Populus, Abies, Pinus, and the like. In some embodiments, the lignocellulosic material comprises a wood or plant flour. In a nonlimiting embodiment, the wood flour is maple wood flour. In some embodiments, the plant flour is soy flour. In some embodiments, the method of growing a mycological biopolymer comprises providing a substrate comprising, consisting of, or consisting essentially of, the lignocellulosic material.

In some embodiments, the natural substrate for supporting growth of the mycological biopolymer is a cellulosic material. As used herein, “cellulosic material” refers to biomass comprising primarily of cellulose. In some embodiments, the cellulosic material is a lignin-free material. Nonlimiting examples of a cellulosic material include plant fibers such as derived from cotton (Gossypium sp.), hemp (Cannabis sp.), flax (Linum Sp.), and jute (Corchorus Sp.). Other nonlimiting examples include pet bedding, paper, cardboard, card stock, cotton, linen and/or textile. In some embodiments, the method of growing a mycological biopolymer comprises providing a substrate comprising, consisting of, or consisting essentially of, the cellulosic material.

In some embodiments, the natural substrate for supporting growth of the mycological biopolymer is an inorganic material, in some further embodiments, the inorganic material is a mineral or mineral based material. Nonlimiting examples of a mineral or mineral-based material include vermiculite, perlite, soils, chalk, gypsum, clay (optionally in the form of bead), sand, rockwool, expanded clay, growstones, or the like. In some embodiments, the mineral or mineral-based substrate is a lignin-free material. In some embodiments, the method of growing a mycological biopolymer comprises providing a substrate comprising, consisting of, or consisting essentially of, the inorganic material, such as the mineral or mineral-based material.

In other embodiments, the substrate supporting growth of the mycological biopolymer is a synthetic material. In some embodiments, the synthetic material is a plastic. In some embodiments, the synthetic material is a synthetic polymer. In some embodiments, the synthetic polymer is a synthetic organic polymer. In some embodiments, the synthetic organic polymer is a polyethylene, a polypropylene, a polyvinyl chloride, a polystyrene, a polyacrylate, a nylon, a polytetrafluoroethylene (e.g., Teflon™), a polyamide, a polyester, a polysulfide, a polycarbonate, a polythene or a polyurethane. In some embodiments, the synthetic organic polymer contains one or more heteroatoms (e.g., nitrogen and/or sulfur), including but not limited to a polyamide, a polyester, a polyurethane, a polysulfide or a polycarbonate. In some further embodiments, the synthetic organic polymer is a polyethylene, or more particularly, a high-density polyethylene or a low-density polyethylene. In some other embodiments, the synthetic organic polymer is a polyurethane, or more particularly, a thermoplastic polyurethane. In some embodiments, the synthetic organic polymer is a nylon, or more particularly nylon 6 or nylon 6,6. In some embodiments, the synthetic material is obtained from recycled materials. In some embodiments, the method of growing a mycological biopolymer comprises providing a substrate comprising, consisting of, or consisting essentially of, the synthetic material.

In yet other embodiments, the substrate supporting growth of the mycological biopolymer is an artificial material. Nonlimiting examples of an artificial material include an aliginate (e.g., an alginate salt such as sodium alginate), rayon (e.g., rayon fiber, such as Viscose), agar or agar-agar, and the like. In some embodiments, the method of growing a mycological biopolymer comprises providing a substrate comprising, consisting of, or consisting essentially of, the artificial material.

In some embodiments, the substrate is non-toxic. As used herein, a non-toxic substrate refers to a substrate that does not inhibit the growth of, or which does not cause the death of the fungus. Thus, the substrate does not contain a toxic substance at a sufficiently high concentration that would inhibit the growth or cause the death of the fungus. In some embodiments, a non-toxic substrate is a substrate that is free of heavy metals, detergents and/or other cytotoxic agents.

In some aspects of the disclosure, the substrate for supporting the growth of the mycological biopolymer is characterized according to a particle size. Various methods of preparing a substrate of a desired particle size are known in the art. Typically, prior to providing the substrate supporting the growth of a mycological biopolymer of the present disclosure, a bulk substrate is provided, which may be sized by methods known in the art. Nonlimiting examples of sizing the substrate include passing a bulk substrate material through a mesh, screen, sieve or the like, use of a shaker table or vibrating screen sorter, etc., grinding, milling, etc., to obtain a substrate of the desired particle size. In some embodiments, the bulk substrate is treated and sized according to methods known in the art to provide the substrate in the form of a wood flour or flour-like material. In some embodiments, the sizing of the substrate to the desired particle size is performed prior to a further processing step, such as a sterilization or pasteurization.

In some embodiments, the substrate particle size is at most about 0.01 inches in diameter. In some embodiments, the particle size is less than about 0.01 inch in diameter; optionally, about 0.007 inch in diameter or less. In some embodiments, the particles are wood or plant flour particles.

In some embodiments, the substrate particle size is at most about 0.125 inch in diameter. In some embodiments, the particle size is less than about 0.125 inch in diameter.

In some embodiments, the substrate particle size is at most about 0.25 inch in diameter. In some embodiments, the particle size is less than about 0.25 inch in diameter.

In some embodiments, the substrate particle size larger, for example, the particle size is at least about 0.25 inch, or greater than 0.25 inch in diameter. In some further embodiments, the particle size ranges from about 0.25 inches to about 2 inches in diameter.

In some embodiments, the substrate particle size refers to a maximum particle diameter. In some embodiments, the particle size refers to a mean particle diameter. In other embodiments, the particle size refers to a median particle diameter.

In some aspects, the substrate for supporting the growth of the mycological biopolymer is characterized according to its form or configuration. In some embodiments, the substrate is a solid. In some embodiments, the substrate is a gel. In some embodiments, the substrate is a liquid, provided that upon or after inoculation with a fungal inoculum, the resulting culture is not a submerged culture. In some embodiments, the substrate is provided as particles, as described herein. In other embodiments, the substrate is provided as a monolithic substrate, such as a contiguous porous solid such as a log, a slab of wood, a solidified porous gel media, or the like. In some more particular embodiments, two or more monolithic substrates are combined (e.g., a log slab penetrating into or through a solidified porous gel media). In other embodiments the substrate is provided as a continuous woven or non-woven textile, such as a rockwool mat, a nonwoven cotton mat, a wood fiber mat, a polyester fiber mat, or the like.

In some embodiments, the substrate is sterilized. In some embodiments, the sterilization is performed prior to inoculation of the substrate with fungal inoculum. Nonlimiting examples of substrate sterilization methods include heat sterilization, steam sterilization (such as autoclaving) or exposure to electromagnetic radiation (such as but not limited to UV irradiation, electron irradiation, gamma irradiation or x-ray irradiation).

In some embodiments, the substrate comprises one or more nutritional agents, without requiring the addition of a further nutritional supplement. Thus, in some aspects, the one or more nutritional agents is inherently present in the substrate obtained or sourced for its intended use in supporting the growth of the mycological biopolymer. In some embodiments, the one or more nutritional agents is one or more organic components (e.g. components or compounds containing carbon oxygen and optionally nitrogen), including but not limited to one or more lipids, simple and/or complex carbohydrates and/or proteins. In some embodiments, the one or more nutritional agents present in the substrate includes one or more mineral(s), vitamin(s), coenzyme(s), element(s), and the like.

In some aspects, the method of growing a mycological biopolymer of the present disclosure comprises providing one or more nutritional supplements to supplement any nutritional value that may be present in the substrate. In some embodiments, the nutritional supplement includes lignocellulosic materials having high fat content, such as seeds. Thus, in some embodiments, the nutritional supplement comprises seed(s), seed husks or both (e.g., sunflower, walnut, poppy seed, etc.). As noted above, in some embodiments, the seeds and/or seed husks are provided as components of the substrate.

In some aspects, the method of growing a mycological biopolymer of the present disclosure comprises providing a fun gal inoculum comprising a fungus. In some embodiments, the fungus is a species of the genus Amillaria, Agaricus, Agrocybe, Ceriporiopsis, Cerioporus, Chlorociboria, Cordyceps, Daldinia, Flammulina, Fomes, Ganoderma, Hericium, Hypomyas, Hypoxylon, Hypsizygous, Inonotus, Laetiporus, Lentinula, Lentinus, Morchella, Omphalotus, Ophiocordyceps, Oxyporus, Phanerochaete, Picnoporus, Pleurotus, Polyporellus, Polyporus, Schizophyllum, Scytalidium, Trametes, Tuber or Xylaria. In some more particular embodiments, the fungus is a species of the genus Flammulina Ganoderma, Inonotus, Lentinula, Morchella or Trametes. In some preferred embodiments, the fungus is a species of the genus Ganoderma. In an even more particular embodiment, the fungus is Ganodermatsugae, Ganoderma resinaceum or G. lucidum. In a more preferred embodiment, the fungus is Ganoderma lucidum. In some other preferred embodiments, the fungus is a species of the genus Morchella. In yet some other preferred embodiments, the fungus is a species of the genus Flammulina.

In some aspects, the method of growing a mycological biopolymer of the present disclosure optionally comprises providing one or more additives. In some embodiments, the substrate is doped with a specific compound, including but not limited to a nitrite, a nutritional supplement, a dietary supplement, a preservative, an antimicrobial agent, a mineral or a drug. The specific compound may then be bio-accumulated by the fungal mycelium tissue and may further become a functional component of the topical applicator containing the mycological biopolymer. In a more particular embodiment, the topical applicator is a bandage or wound dressing, and the additive is an antimicrobial agent. The present disclosure this provides for a method of impregnating a bandage or wound dressing with the antimicrobial agent. In a further nonlimiting example, the antimicrobial agentis a tannin, cinnamaldehyde, AgCl, ZnCl or a sorbate.

In some embodiments, the additive is a nutritional agent or supplement, which is optionally added to the substrate to support mycelial growth during the production of the mycological biopolymer. In some aspects, the substrate (e.g., an agar media) is formulated through the addition of nutrition, including but not limited to supplements or minerals. In a particular embodiment the supplement is forskolin. In another particular embodiment, the supplement is 10-oxo-rans-8-decenoic acid. Such additives may support mycelial growth. In another embodiment, an alternative or additional additive is a drug.

In some embodiments, the mixture further comprises a nutritional agent. In such embodiments, the method of preparing the mycological biopolymer of the disclosure further comprises preparing a blend, the blend containing the substrate, as disclosed herein, and the nutritional agent. In a nonlimiting example of a method of the disclosure, a fungal inoculum is added to the blend to provide the mixture. Further agents or additives such as those disclosed herein may be added to the mixture.

In some further embodiments, the method further comprises sterilizing the substrate or blend prior to providing the mixture. In some embodiments, method comprises sterilizing the substrate or blend using heat sterilization, steam sterilization, or irradiation with electromagnetic radiation, such as electron radiation, gamma radiation, x-ray radiation, ultraviolet (UV) or UV-visible radiation.

In some embodiments, the method further comprises placing the mixture in a tool. A suitable tool includes a tool is essentially as described in US2015/0033620, the entire content of which is hereby incorporated by reference in its entirety. In some embodiments, a suitable tool is an incubation chamber. In other embodiments, the method comprises placing the mixture on a planar surface. Nonlimiting examples of a planar surface for placing the mixture include a tray, a sheet, a table, a conveyer belt and the like. In another aspect, the method comprises exposing the mixture contained in the tool or placed on the planar surface to growth environmental conditions, thereby initiating an incubation period. In yet further aspects, the method further comprises incubating the mixture as a solid-state culture for a period of time in the growth environment.

In some aspects, the method excludes submerging the culture in a liquid.

In yet further aspects, the method comprises terminating the incubation prior to the fungus forming a visible fungal fruiting body. Thus, in some embodiments, the method comprises terminating the incubation prior to the formation of a visible stipe, pileus, gill or pore structure associated with the mycological biopolymer. In some embodiments, terminating the incubation comprises modifying one or more of the growth environmental conditions.

In some embodiments, the method of making the mycological biopolymer further comprises drying the mycological biopolymer. In some embodiments, the mycological biopolymer is dried after the termination of the growth. In some embodiments, the mycological biopolymer is dried at a temperature and for a period that removes at least a portion of residual water. In some embodiments, the drying of the mycological biopolymer does not detrimentally impact the protein content of the mycological biopolymer. Drying methods include but are not limited to convective drying, conductive drying, drying under ambient conditions, and freeze drying (lyophilizing). In some embodiments, the mycological biopolymer is dried at a temperature suitable for removing moisture; for example, about 100 to about 180° F.; optionally, about 110° F. In some embodiments, the mycological biopolymer is dried until the moisture content is less than about 20%, less than about 15% or preferably, less than about 10% (w/w) of the total mass of the dried mycological biopolymer.

In still other aspects, the methods of the disclosure provide mycological biopolymers suitable for use as topical applicators, as disclosed herein.

In some embodiments, such as when the mycological biopolymer comprises residual substrate, the method optionally further comprises removing the substrate or a decomposition product thereof from the mycological biopolymer.

In some embodiments, the present disclosure provides for imparting a form or shape to the mycological biopolymer to provide a topical applicator having said form or shape. In some embodiments, the method comprises cutting and/or compressing the mycological biopolymer into the desired form or shape. In some embodiments, the cutting comprises hand cutting, die cutting, laser cutting, water jetting, computer numerical control (CNC) machined-knife cutting, or a combination thereof. In some embodiments, the mycological biopolymer, optionally containing one or more additional ingredients, is formed or shaped into a sheet, a circular disc, a triangle, a cylinder, a rectangle, a cone, or any other 3-dimensional molded form or custom design. In some more particular embodiments, the mycological biopolymer, optionally containing one or more additional ingredients, is formed or shaped into a mask (for example, a face mask, a hand mask or foot mask); a finger space or toe spacer; a skin cleansing foam; an ear plug; a wipe (for example, a disinfectant wipe and/or baby wipe); a bandage; a burn or wound dressing; or a cosmetic (or other product) applicator or remover (for example for application or removal of a cosmetic to the skin or lips).

In some aspects, the present disclosure provides for methods of incorporating one or more additional substances into the mycological biopolymer or a topical applicator comprising the mycological biopolymer. Generally, an additional substance contained by the topical applicator is provided to enhance the health or beauty of a subject; such benefits can be achieved once the topical applicator has been applied to an application region of the subject. Thus, in preferred embodiments, the additional substance(s) is a health or beauty product or ingredient, or more particularly, a topical health or beauty product or ingredient. As an alternative or further benefit, an additional substance is optionally incorporated into the mycological biopolymer, or the topical applicator comprising the mycological biopolymer, in order to tune its physical characteristics (e.g., hydrophobicity), to inhibit the growth of microbial contaminants (e.g., to extend its shelf life), and/or to enhance or provide for the uptake and/or transfer of a health or beauty product or ingredient from the topical applicator to the application region of a subject. As disclosed herein, nonlimiting examples of topical health or beauty products and ingredients for incorporation into a topical applicator of the present disclosure include an anesthetic agent, an analgesic, an antimicrobial, an antibiotic, an antiseptic, an anti-inflammatory, an exfoliant, a cosmetic, a sunscreen, a sun lotion, a moisturizer, a topical burn treatment agent, a cleanser, an astringent, a toner, a chelator, an anti aging product, an anti-acne agent, an anti-coagulant; a protein; a signaling molecule; a sugar (e.g., a polysaccharide or oligosaccharide, or a derivative or degradation product thereof); a pigment; a vitamin; a nutritional supplement; an oil; a microbe; or a combination thereof.

In some embodiments, the one or more additional substances is incorporated into the mycological biopolymer, or the topical applicator containing the mycological biopolymer, during the manufacture of the topical applicator, and can occur during or after the growth of the mycological biopolymer.

In some embodiments, the one or more additional substances is incorporated after the growth of the mycological biopolymer, i.e., after the incubation of the inoculated growth medium in the growth environment is terminated. The resulting mycological biopolymer, which is optionally predried to a residual moisture content of less than about 10%, is then treated with the one or more additional substances, thereby incorporating the substance(s) into the mycological biopolymer. The method of incorporating the substance(s) into the mycological biopolymer includes any method suitable to achieve this end. In a nonlimiting example, the mycological biopolymer is soaked (by partial or complete immersion) in a formulation containing or consisting of the additional substance(s); the formulation is optionally, a fluid, including but not limited to as a solution, an emulsion or a suspension. In another nonlimiting example, the mycological biopolymer is injected with a formulation containing or consisting of the additional substance(s) (optionally, a fluid). In yet another nonlimiting example, a formulation containing or consisting of the additional substance(s) optionally in the form of a fluid is dropped onto a surface of the mycological biopolymer (for example, by pipette or micropipette or needle). In yet another nonlimiting example, a formulation containing or consisting of the additional substance(s) (optionally, in the form of a fluid, a gel, a cream, a lotion, a suspension or a solid, such as a powder or crystalline particles) is applied to a surface of the mycological biopolymer, for example, spread across the surface of the mycological biopolymer. In yet another nonlimiting example, a formulation containing or consisting of the additional substance(s) (optionally, a solid comprised of particles) is sprinkled or seeded across the surface of the mycological biopolymer. In some embodiments, the formulation contains one or more microorganisms, including probiotics, which can be seeded into the mycological biopolymer. The mycological biopolymer is treated with the additional substance(s) for a period of time sufficient to imbibe the mycological biopolymer with the additional substance(s). Any one of the foregoing processes can be performed manually or by automation. In some further embodiments, the mycological biopolymer is imparted with a desired form or shape, as disclosed herein, before or after the incorporation of the additional substance(s), to provide the topical applicator containing the mycological biopolymer and the one or more additional substances.

In other embodiments, the one or more additional substances (including microorganisms) is incorporated into the mycological biopolymer during the growth of the mycological biopolymer, i.e., prior to terminating the incubation of the inoculated growth medium in the growth environment. Optionally, any of the methods described above for the incorporation of the additional substance(s) after the termination of mycological biopolymer growth can be adopted for use during the growth process.

In some embodiments, the additional substance is a protein, in a more particular embodiment, the protein is a collagen, a silk protein or a neurotoxic protein (e.g., Botulinum toxin or Botox®). In some embodiments, the additional substance is a polysaccharide or oligosaccharide. Nonlimiting examples of a polysaccharide or oligosaccharide include chitin, hyaluronic acid and beta-glucans; and combinations thereof.

In some alternative embodiments, a condition of growth of the mycological biopolymer is modulated, thereby altering the final content of a fungal mycelial metabolite, such as a primary metabolite, including a structural protein or complex carbohydrate (e.g., whereby the modulation increases or decreases the protein and/or carbohydrate content in the mycological biopolymer, and/or results in the deposition of metabolic or chemical degradation products or precursors of the structural protein or carbohydrate into the mycological biopolymer). Thus, additional substances can be incorporated into a topical applicator of the disclosure by such modulation methods.

In some embodiments, the substance(s) is a protein. In some embodiments, a mycological biopolymer is grown such that at least one protein is induced and incorporated into the structure of the mycological biopolymer. In some embodiments, the protein is induced using environmental or incubation stimuli, nutritional inducers within the growth media or substrate, through genetic engineering to either upregulate native genes or knock out those that may inhibit the production of the protein, or a combination thereof.

In some embodiments, the protein is hydrophobin. Hydrophobin is a primary metabolite of the mycological biopolymers of the present disclosure; its content may be modulated to tune the properties of the topical applicator comprising the mycological biopolymer. Methods of modulating hydrophobin content of a mycological material are disclosed, for example, in U.S. Ser. No. 16/363,052, published as U.S. 2019/0322997 A1, the entire contents of which is hereby incorporated by reference in its entirety, in some embodiments, the hydrophobin imparts the topical applicator with benefits including repulsion of water; absorption of oil, durability, reusability; or a combination thereof.

In some embodiments, the protein is an enzyme. In some embodiments, a topical applicator comprising the enzyme, when applied to an application region of a subject, provides benefits such as degrading or eliminating skin oils, bolstering collagen production, and/or degrading or inhibiting the growth of undesirable microbes, such as those that could lead to dermatitis. In some embodiments, the enzyme is a chitin deacetylase. As disclosed herein, chitin is a primary metabolite of mycological biopolymers of the present disclosure. In some embodiments, a chitin deacetylase, when incorporated into a mycological biopolymer of the present disclosure, converts a portion of the chitin into chitosan, which has antimicrobial properties. Thus, the incorporation of chitin deacetylase into the mycological biopolymer deposits chitin into the mycological biopolymer, which can serve to inhibit the growth of microbial contaminants, thereby extending the shelf life or reusability of the topical applicator comprising the mycological biopolymer so produced, and/or providing for the transfer of chitin to an application region of a subject once the topical applicator contacts the application region. Other benefits of the incorporation of chitin deacetylase include its ability to sequester metal salts through carboxylation or phosphorylation, and to functionalized within additional protein-based polymers such as collagen or silk (see US2019/0338240, the entire content of which are hereby incorporated by reference in in its entirety). Other methods of modulating the chitin content of a mycological material are disclosed, or example, in U.S. Ser. No. 16/363,052, published as U.S. 2019/0322997, the entire content of which is hereby incorporated by reference in its entirety.

In some embodiments, the substance(s) is a complex carbohydrate (e.g. an oligosaccharide or polysaccharide). In some embodiments, a mycological biopolymer is grown such that at least one complex carbohydrate is induced and incorporated into the structure of the mycological biopolymer. In some embodiments, the complex carbohydrate is induced using environmental or incubation stimuli, nutritional inducers within the growth media or substrate, through genetic engineering to either upregulate native genes or knock out those that may inhibit the production of the complex carbohydrate, or a combination thereof. In some embodiments, the complex carbohydrate is a beta-glucan, which is a primary metabolite of the mycological biopolymers of the present disclosure. The mycological biopolymer beta-glucan content can be modulated to tune the properties of the topical applicator comprising the mycological biopolymer. Other methods of modulating the beta-glucan content of a mycological material are disclosed, for example, in U.S. Ser. No. 16/363,052, published as US 2019/0322997, the entire content of which is hereby incorporated by reference in its entirety. The topical applicator can be moistened in water to solubilize some of the beta-glucan content within the mycological biopolymer, which may be transferred to the application region of a subject when the topical applicator makes contact with the application region.

In some other embodiments, the substance is a secondary metabolite, including but not limited to a pigment. Modulating a condition of growth of the mycological biopolymer can alter the final content of the fungal mycelial secondary metabolite in the mycological biopolymer. Methods of modulating the melanin content of a mycological material are disclosed, for example, in U.S. Ser. No. 16/363,052, published as US 20190132297, the entire content of which is hereby incorporated by reference in its entirety.

In some embodiments, the additional substance is a microorganism, or one or more substances produced by a microorganism. In some embodiments, the microorganism is a probiotic. In some embodiments, the microorganism is one that produces and excretes desired compounds into the mycological biopolymer, such as anti-inflammatory agents, collagen, silk protein, and other skin care ingredients. In some embodiments, the method of incorporating microorganisms into a mycological biopolymer of the present disclosure comprises misting (or other means of deposition of) the fungal-inoculated growth medium with a fluid (e.g., a suspension) containing select microorganisms, such as bacteria, yeast, or other desired microbes, thereby depositing one or more microbial communities across the surface of the growing mycological biopolymer. Consequently, the deposited organisms or communities are incorporated into the growing mycological biopolymer. The misting with the selected microorganisms occurs during the mycelial growth period. In some embodiments, the misting occurs at periodic intervals, optionally ranging from each minute to each day. By sequencing the time and interval of misting or deposition, microbial communities may be resolved into distinct and spatially separated layers in the mycelial mat, or more generally, the relative concentration of the microbial communities can be modulated within the mycelial mat. In some embodiments, the deposited microbial community continues to grow and proliferate as the mycological biopolymer forms. Said community can excrete one or more desired biological compounds into the mycological biopolymer, such as collagen protein, anti-inflammatory agents, signaling molecules, pigments, or other beneficial additives commonly used in the health or cosmetics industry. In some alternative embodiments, the microbial community enters a resting states and as such can optionally be reactivated at a later time via one or more environmental triggers, such as heat, water, pH change, time and/or motion. At the end of the incubation period, the mycological biopolymer comprising the active or resting microbial community is extracted from the incubator and dried at such a temperature to preserve the bioactivity of the imbedded microorganisms or bioactive compounds secreted during the growth process. In a further embodiment, a topical applicator comprising the select microorganisms is applied to/makes contact with an application region of a subject, thereby transferring the beneficial microbial community, and/or agents produced by said community, to the application region, and consequently improving the health or appearance of the subject's skin.

### Exemplary Mycological Biopolymers for Use as Topical Applicators

### Embodiment A

A mycological biopolymer suitable for direct use or in the manufacture of a topical applicator of the present disclosure is prepared by incubating a fungal inoculum containing a fungus, as disclosed herein, with a substrate (optionally containing additional nutritional supplement) as a solid-state culture in a growth environment that supports the growth of mycelial tissue, without the formation of a visible fruiting body, such as a stipe, pileus, gill or pore structure.

The growth environment is characterized as follows:

the atmosphere contains oxygen and further contains carbon dioxide at a level ranging from about 3% (v/v) to about 7% (v/v), or from about 5% (v/v) to about 7% (v/v);

the temperature ranges from about 85° F. to about 95° F., or from about 85° F. to about 90° F.; and

the relative humidity is at least about 95%, for example, about 99%.

The incubation time in the growth environment is sufficient to produce a mat of aerial mycelial tissue.

### Embodiment B

Embodiment A, and:

The growth environment includes a directed flow of air. The directed air flow is a horizontal airflow at rate in the range of about 250 feet per minute to about 500 feet per minute.

The fungus is a species of the genus Ganoderma, optionally, Ganoderma lucidum.

The resulting mycelial tissue is removed from the substrate to provide a panel consisting essentially of the mycological biopolymer having a density of greater than 3 pounds per cubic foot (as measured after drying to a moisture content of less than about 10%).

The mycological biopolymer can have a tensile strength of about 25 to about 35 psi.

The mycological biopolymer, having been tuned for high density via the high rate of horizontal air flow, can be converted into a variety of topical applicators, such as makeup applicators which require greater density and stiffness for use.

### Embodiment C

Embodiment A, and:

The growth environment includes a directed flow of air. The directed air flow is a horizontal air flow at rate in the range of about 100 feet per minute to about 250 feet per minute.

The fungus is a species of the genus Ganoderma, optionally, Ganoderma lucidum.

The resulting mycelial tissue is removed from the substrate to provide a panel consisting essentially of the mycological biopolymer having a density in a range of about 0.8 to about 3 pounds per cubic foot (as measured after drying to a moisture content of less than about 10%), with a high degree of uniformity.

The mycological biopolymer can have a tensile strength of about 10 to about 20 psi.

The mycological biopolymer, having been tuned for moderate density via the intermediate rate of horizontal air flow, can be converted into a variety of topical applicators, such as face masks which require uniformity and sufficient density to be successfully sliced thinly.

### Embodiment D

Embodiment A, and:

The growth environment includes a directed flow of air. The directed air flow is a horizontal air flow at rate of less than 100 feet per minute. Alternatively, the growth environment does not include directed air flow.

The fungus is a species of the genus Ganoderma, optionally, Ganoderma lucidum.

The resulting mycelial tissue is removed from the substrate to provide a panel consisting essentially of the mycological biopolymer having a density in a range of about 0.5 to about 2 pounds per cubic foot (as measured after drying to a moisture content of less then about 10%).

The mycological biopolymer, having been tuned for lower density via the lower rate of horizontal air flow, can be converted into a variety of topical applicators that are preferably made from softer, lower density materials, such as wipes, for example, for the removal of makeup or other products from the skin or lips.

### Embodiment E

Embodiment A, and:

The fungus is a species of the genus Morchella.

The resulting mycelial tissue is removed from the substrate to provide a panel consisting essentially of the mycological biopolymer having a density of less than about one pound per cubic foot (as measured after drying to a moisture content of less than about 10%). The material is extremely soft.

This soft and ultralow density mycological biopolymer is useful for the softest applicator or wipe products, such as baby wipes.

Some nonlimiting embodiments of the disclosure are listed below.
1. A topical applicator comprising a structural matrix, wherein the structural matrix comprises a mycological biopolymer.
2. The topical applicator of embodiment 1, wherein the topical applicator is suitable for use in applying a health or beauty product to an application region of a subject.
3. The topical applicator of embodiment 1 for use in applying a health or beauty product to an application region of a subject.
4. The topical applicator of embodiment 2 or 3, wherein the application region is the subject's skin or lips.
5. The topical applicator of any one of embodiments 1 to 4, wherein the mycological biopolymer is produced by a method comprising:
   providing a growth medium comprising a fungal inoculum and a substrate, said substrate optionally further comprising a supplemental source of nutrition, and said fungal inoculum comprising a fungus; and
   incubating the growth medium, as a solid-state culture for a period of time in a growth environment, wherein the growth environment has a relative humidity, a temperature, carbon dioxide (CO₂) and oxygen (O₂) sufficient to support growth of the mycological biopolymer without the formation of a visible fruiting body; thereby providing the mycological biopolymer.
6. The topical applicator of embodiment 5, wherein the growth environment carbon dioxide level is within a range of about 3% (v/v) to about 7% (v/v).
7. The topical applicator of embodiment 6, wherein the growth environment carbon dioxide level is within the range of about 5% (v/v) to about 7% (v/v).
8. The topical applicator of any one of embodiments 5 to 7, wherein the growth environment relative humidity is at least about 95%.
9. The topical applicator of embodiment 8, wherein the growth environment relative humidity is about 99%.
10. The topical applicator of any one of embodiments 5 to 9, wherein the growth environment has a temperature ranging from about 55° F. to about 100° F.
11. The topical applicator of embodiment 10, wherein the growth environment has a temperature ranging from about 85° F. to about 95° F., or from about 85° F. to about 90° F.
12. The topical applicator of any one of embodiments 5 to 11, wherein the fungus is a species of a genus selected from the group consisting of Flammulina Ganoderma, Inonotus, Lentinula, Morchella and Trametes.
13. The topical applicator of any one of the preceding embodiments, wherein the mycological biopolymer has a density ranging from about 0.1 to about 5 pounds per cubic foot.
14. The topical applicator of any one of the preceding embodiments, wherein the mycological biopolymer has an open volume ranging from about 70% to about 99%, about 75% to about 99%, about 80% to about 99%, or about 80% to about 92% (v/v).
15. The topical applicator of embodiment 13 or 14, wherein the mycological biopolymer has a density of at least about 3 pounds per cubic foot.
16. The topical applicator of embodiment 15, wherein the growth environment has a horizontal directed air flow, said airflow having a rate ranging from about 250 feet per minute to about 500 feet per minute, or about 275 feet per minute to about 500 feet per minute.
17. The topical applicator of embodiment 15 or 16, wherein the mycological biopolymer has a tensile strength ranging from about 25 to about 35 psi.
18. The topical applicator of embodiment 13 or 14, wherein the mycological biopolymer has a density ranging from about 0.8 to about 3 pounds per cubic foot.
19. The topical applicator of embodiment 18, wherein the growth environment has a horizontal directed air flow, said air flow having a rate ranging from about 100 feet per minute to about 250 feet per minute, or about 110 to about 250 feet per minute.
20. The topical applicator of embodiment 18 or 19 wherein the mycological biopolymer has a tensile strength ranging from about 10 to about 20 psi.
21. The topical applicator of embodiment 13 or 14, wherein the mycological biopolymer has a density ranging from about 0.5 to about 2 pounds per cubic foot.
22. The topical applicator of embodiment 21, wherein the growth environment has a horizontal directed air flow, said air flow having an air flow rate of at most about 100 feet per minute, or less than about 100 feet per minute.
23. The topical applicator of any one of embodiments 12 to 22, wherein the fungus is a species of the genus Ganoderma.
24. The topical applicator of embodiment 23, wherein the fungus is Ganoderma lucidum.
25. The topical applicator of embodiment 13 or 14, wherein the mycological biopolymer has a density of less than about 1 pound per cubic foot.
26. The topical applicator of embodiment 25, wherein the fungus is a species of the genus Morchella.
27. The topical applicator of any one of embodiments 5 to 26, wherein the growth medium is incubated in the growth environment as the solid-state culture for a period of time of up to about 3 weeks.
28. The topical applicator of embodiment 27, wherein the period of time is from about 4 days to about 14 days.
29. The topical applicator of embodiment 28, wherein the period of time is about 9 days.
30. The topical applicator of any one of the preceding embodiments, wherein the mycological biopolymer has a fluid retention capacity of up to about 20-fold of the dry mass of the mycological biopolymer.
31. The topical applicator of any one of embodiments 1 to 11, wherein the mycological biopolymer comprises hyphal filaments having a thickness ranging from about 0.5 micron to about 10 microns, or from about 0.5 micron to about 6 microns.
32. The topical applicator of any one of embodiments 1 to 11, wherein the mycological biopolymer has a modulus of elasticity ranging from about 1 to about 100 psi.
33. The topical applicator of embodiment 32, wherein the mycological biopolymer has a modulus of elasticity ranging from about 30 to about 60 psi, about 35 to about 60 psi, or about 35 to about 55 psi.
34. The topical applicator of any one of embodiments 5 to 14, wherein the mycological biopolymer provided by the method has a mat height ranging from about 0,125 inch to about 3 inches.
35. The topical applicator of any one of embodiments 1 to 11, wherein the mycological biopolymer has a hydrophobicity characterized by a contact angle ranging from about 90 to about 150 degrees, about 100 to about 130 degrees, or about 110 to about 125 degrees.
36. The topical applicator of any one of embodiments 1 to 11, wherein the mycological biopolymer has a chitin content ranging from about 5% to about 35%, about 5% to about 25%, or about 5% to about 15%, about 8 to about 12%, or about 10% (w/w) of the dry mass of the mycological biopolymer.
37. The topical applicator of any one of embodiments 1 to 11, wherein the mycological biopolymer has a protein content ranging from about 1% to about 25%, about 2% to about 20%, about 5% to about 10%, or about 6% to about 9% (w/w) of the dry mass of the mycological biopolymer.
38. The topical applicator of any one of the preceding embodiments, wherein the structural matrix consists of, or consists essentially of the mycological biopolymer.
39. The topical applicator of any one of the preceding embodiments, wherein the topical applicator consists of the mycological biopolymer.
40. The topical applicator of any one of embodiments 1 to 38, wherein the topical applicator further comprises an additional substance.
41. The topical applicator of embodiment 40, wherein the additional substance is a health or beauty product.
42. The topical applicator of embodiment 41, wherein the additional substance is a health or beauty product suitable for topical administration.
43. The topical applicator of embodiment 40 or 41, wherein the additional substance is an anaesthetic, an analgesic, an antimicrobial, an antibiotic, an antiseptic, an anti-inflammatory, an exfoliant, a cosmetic, a sunscreen, a sun lotion, a moisturizer, a topical burn treatment agent, a cleanser, an astringent, a toner, a chelator, an anti-aging product, an anti-acne agent, an anticoagulant, a protein, a signaling molecule, a complex carbohydrate, a pigment, a vitamin, a nutritional supplement, an oil or a microbe; or a combination thereof.
44. The topical applicator of embodiment 43, wherein the additional substance is a protein.
45. The topical applicator of embodiment 44, wherein the protein is collagen,
46. The topical applicator of embodiment 43, wherein the additional substance is a complex carbohydrate.
47. The topical applicator of embodiment 46, wherein the complex carbohydrate is hyaluronic acid, beta-glucan, or a combination thereof.
48. The topical applicator of embodiment 47, wherein the beta-glucan is water soluble.
49. The topical applicator of embodiment 40 or 41 wherein the additional substance is added to the mycological biopolymer after the growth of the mycological biopolymer is terminated.
50. The topical applicator of embodiment 43, wherein the additional substance is a microbe.
51. The topical applicator of embodiment 50, wherein the microbe is added during the growth of the mycological biopolymer.
52. The topical applicator of any one of the preceding embodiments, wherein the topical applicator is provided in the form of a sheet, a circular disc, a triangle, a cylinder, a rectangle or a cone, or any other 3-dimensional molded form or custom design.
53. The topical applicator of any one of embodiments 1 to 51, wherein the topical applicator is a mask, a finger spacer, a toe spacer, a cleansing foam, a wipe, an ear plug, a product applicator or a product blender.
54. The topical applicator of any one of embodiments 1 to 53 for use in the application of a beauty product.
55. The topical applicator of embodiment 64, wherein the beauty product is a cosmetic.
56. The topical applicator of any one of embodiments 1 to 53 for use in the application of a health product.
57. The topical applicator of embodiment 56, wherein the topical applicator is a bandage, a burn dressing or a wound dressing.
58. The topical applicator of any one of embodiments 1 to 39, wherein the topical applicator is biodegradable.
59. The topical applicator of any one of embodiments 5 to 58, wherein the growth medium substrate is a lignocellulosic substrate.
60. The topical applicator of embodiment 59, wherein the lignocellulosic substrate is an agricultural waste product selected from the group consisting of corn stover, kenaf pith, canola straw and wheat straw.
61. The topical applicator of embodiment 59, wherein the lignocellulosic substrate is not an agricultural waste product.
62. The topical applicator of embodiment 61, wherein the lignocellulosic substrate is soy flour or maple wood flour.
63. The topical applicator of any one of embodiments 5 to 58, wherein the growth medium substrate is a cellulosic substrate.
64. The topical applicator of embodiment 63, wherein the cellulosic substrate is a lignin-free material.
65. The topical applicator of any one of embodiments 5 to 58, wherein the growth medium substrate is an inorganic material.
66. The topical applicator of embodiment 65, wherein the inorganic material is vermiculite, perlite, soils, chalk, gypsum, clay, sand, rockwool, expanded clay or growstones.
67. The topical applicator of embodiment 5, wherein the fungus is genetically engineered to overexpress a chitin deacetylase (DCA) gene.
66. The topical applicator of embodiment 5, wherein the fungus is genetically engineered to overexpress hydrophobins.
69. The topical applicator of embodiment 5, wherein the fungus is of the genus Ganoderma genetically engineered to overexpress the genes BGS1 and BGS2 that encode the two β-1,3-glucan synthases therein.
70. A method of enhancing the health a subject, the method comprising using a topical applicator of any one of embodiments 1 to 69 to apply a health product to an application region of a subject in need thereof.
71. A method of enhancing the aesthetic appearance of a subject, the method comprising using a topical applicator of any one of embodiments 1 to 69 to apply a beauty product to an application region of a subject in need thereof.

### EXAMPLES

The following sets forth several examples of topical applicators comprising mycological biopolymers of the present disclosure.

### Example A

Preparation of mycological biopolymer. Mycological biopolymer was obtained by inoculating growth matrix (containing corn stover substrate (previously sized with a ⅛″ mesh) supplemented with poppy seeds, maltodextrin, calcium sulfate) with inoculum containing Ganoderma sp. The inoculated growth matrix was incubated for 9 days at a temperature of 85° F. to 90° F. in an environment containing 5% (v/v) carbon dioxide at 99% relative humidity.

The mycological biopolymer was extracted from the growth medium and dried for 18 hours at 110° F., after which the residual moisture content was less than about 10% (w/w) of the total mass of the mycological biopolymer.

### Example B

Hyphal Filament Dimensions.

Diameter. Mycological biopolymer was obtained essentially as described in Example A. Sections were sliced along the thickness of the mycological biopolymer and embedded in epoxy resin. The epoxy embedded mycological biopolymer was then microsectioned and optically analyzed via autofluorescence to determine the hyphal diameter of the mycological biopolymer tissue. The results indicated that the diameter of the hyphae ranged from 0.25 micron to 5 microns. The majority of the hyphae had a diameter of between about 0.5 micron and 2 microns.

Radius. Mycological biopolymer was obtained essentially as described in Example A. Sections were sliced along the thickness of the mycological biopolymer and analyzed by scanning electron microscopy (SEM) and radius of the hyphae of the mycelial tissue was determined. The results indicated that the mean radius of the hyphae was 0.42 micron, and that the hyphae radii ranged from 0.125 micron to 1.6 microns.

### Example C

Open volume. Mycological biopolymer was obtained essentially as described in Example A. The open volume (porosity) of the mycological biopolymer was measured by a variety of methods.

In one experiment, sections of the mycological biopolymer were sliced along its thickness and analyzed by fluid saturation. The open volume of the mycological biopolymer was determined to be between 84% and 93% (v/v).

In another experiment, the mycological biopolymer was embedded with a clear epoxy resin and was ground to a thin section using common thin sectioning techniques. This section was then imaged on a light microscope and the images were analyzed for open volume percentage. The biopolymer was determined to be about 80% to 99% (v/v).

In yet other experiments, the mycological biopolymer was inspected either by scanning electron microscopy (SEM), confocal or micro-computed tomography (CT) scanning techniques and again analyzed for open volume percentage. The biopolymer was determined to be about 80% to about 88% (v/v).

### Example D

Fluid retention capacity. Mycological biopolymer was obtained essentially as described in Example A.

In one experiment, the biopolymer was die cut and immersed in excess saline solution. Saturated material was massed and found to have absorbed on average 13 times the dry mass of the mycological biopolymer material.

### Example E

Hydrophobicity. The mycological biopolymer prepared as described in Example A was analyzed by optical tensiometry and found to have hydrophobic contact angles between 110 and 124°. The fungal protein hydrophobin is predominately responsible for this property.

### Example F

Hydrophilicity. A mycological biopolymer prepared as described in Example A and having a native hydrophobic contact angle between 110-124°, was immersed in primary alcohol solution (70-100% (v/v)) for a period of over 3 weeks and then convection dried at 110° F. The mean contact angle after treatment was determined to be 73.7° via optical tensiometry.

### Example G

Modulus of Elasticity. A mycological biopolymer prepared essentially as described in Example A, was determined to have a modulus of elasticity ranging between 35 and 45 psi as measured via ASTM D638.

### Example H

Chitin content. A mycological biopolymer prepared essentially as described in Example A, was determined to have a chitin content of about 10% (w/w) of its dry mass, calculated via analysis of Fourier transform infrared (FT-IR) spectral data at the 1655 cm⁻¹ peak.

### Example 1

1. A mycological biopolymer is grown on a nutritional media such that a beta-glucan matrix is induced as a portion of the open cellular structure of the biomaterial. The beta-glucans can be induced using environmental or incubation stimuli, nutritional inducers within the growth media or substrate, through genetic engineering to either upregulate native genes or knock-out those that may inhibit the production of beta-glucans, or a combination thereof.

2. The mycological biopolymer is harvested from the nutritional media. This is accomplished by either peeling or cutting the mycelial mat away from the media.

3. The mycological biopolymer is dried at a temperature for a period of time sufficient to inactivate the growth of the fungus.

4. The dried mycological biopolymer material is sliced into 2 mm sections across the horizontal or vertical plane of the material.

5. The thin sections of mycological biopolymer are die cut into a two-dimensional geometry of a skincare face mask.

6. The face mask can be moistened in water, such as tap water, to solubilize some of the beta-glucan content within the mycological biopolymer, and the mask placed directly on the skin.

7. The face mask is discarded after use, ideally in a plant-based compost for biodegradation.

### Example 2

1. A mycological biopolymer is grown such that at least one protein is induced upon the open cellular structure of the biomaterial. The proteins can be induced using environmental or incubation stimuli, nutritional inducers within the growth media or substrate, through genetic engineering to either upregulate native genes or knock-out those that may inhibit the production of proteins, or a combination thereof.

2. Same as “1”. wherein the induced protein is one or more enzymes that could serve to:
a. Degrade and eliminate skin oils.
b. Bolster the production of collagen.
c. Target non-desirable microbes that could lead to dermatitis.

3. Same as “1.” wherein the induced protein is a hydrophobin that could serve to:
a. Repel water.
b. Bolster reusability and durability.
c. Sorb oil while repelling water.

4. Same as “1.” wherein the induced protein is an enzyme within the family of chitin deacetylase, which functionalizes the chitin within the fungal cell wall.
a. Creates chitosan, which provides antimicrobial properties.
b. Can sequester metal salts through carboxylation or phosphorylation.
c. Can be functionalized within additional protein-based polymers such as collagen or silk.

5. The mycological bipolymer is harvested from the nutritional media. This is accomplished by either peeling or cutting the tissue away for the media.

6. The mycological biopolymer is dried at a temperature and for a period that removes the residual water but does not detrimentally impact the protein content. Drying methods could include but are not limited to convective drying and freeze drying (lyophilizing).

7. The mycological biopolymer is sliced into 12 to 18 mm sections across the horizontal or vertical plane of the material.

8. The sliced sections of the mycological biopolymer are die cut into the circular discs, rectangles, and/or triangles for use as cosmetics applications or facial cleansing sponges.

### Example 3

### Process for Incorporating Bio-Active Compounds

1. A mycological biopolymer is grown such that a protein or enzyme, such as chitin deacetylase, is induced upon the open cellular structure of the biomaterial.

2. During incubation at periodic intervals ranging from each minute to each day a mist of dilute solute containing bacteria, yeast, or other selected microbes and microbial communities is deposited across the surface of the growing mycological biopolymer such that the deposited organisms or communities are incorporated into the growing mycological biopolymer.

3. Such communities either
a. Continue to grow within the part as the mycological biopolymer forms
b. Enter a resting state, to be reactivated at a later time via one or more triggers (heat, water, pH change, time, motion)

4. By sequencing the time dimension of misting or deposition at varying intervals a resolution of layer spacing of microbes can be established effectively modulating concentration within the material.

5. Deposited microbes, during the biopolymer growth process, may continue to
a. proliferate; or
b. proliferate and excrete one or more desired biological compounds such as collagen protein, anti-inflammatory agents, signaling molecules, or other beneficial additives commonly found in the cosmetics industry.

6. The material completes its growth cycle and is extracted from the incubator and dried at such a temperature to preserve the bioactivity of the imbedded organisms or bioactive compounds secreted during the growth process.

### Example 4

As in Example 3, wherein microorganisms are selected and embedded into the applicator through either the process steps of 2.5 of infusion such that a micro-biome that improves the health of one's skin is present in the applicator and by application to the face will transfer said microbial community to the surface of the skin.

### Example 5

5A. 1. A mycological biopolymer is grown such that the porosity (open volume) of the matrix is sufficient to imbibe or infuse other ingredients.

2. Steps 2-4 of Example 1 are repeated.

3. Coconut oil, which is solid at room temperature, is heated in a microwave for 30 seconds or until it is fully in a liquid state.

4. The coconut oil is diluted in warm tap water at a 1:1 (v/v) concentration and stirred until sufficiently mixed to form a coconut oil and water emulsion. Other ingredients that are compatible with water and coconut oil can be included in the emulsion at this time as well.

5. One or more pieces of the mycological biopolymer are immersed in the emulsion for a period of 30 seconds.

6. The coconut-oil infused mycological biopolymer is removed from the emulsion and permitted to cure at room temperature or within a refrigerated environment.

7. The mycological biopolymer is now imbibed with coconut oil and optionally other ingredients, and can serve as an applicator to apply the ingredients directly to the skin.

5B. 1. Refined organic coconut oil (about 200 mL) was microwaved for one minute until completely melted.

2. Hot municipal tap water was added to a 500 mL vessel.

3. The melted coconut oil (200 mL) was added to the water and stirred to provide an emulsion.

4. Three Mycoflex™ wedges (about 0.2 g each) were immersed in the emulsion for 20 to 40 seconds.

5. The wedges, imbibed with the coconut oil in water emulsion was allowed to cool and drip drain at room temperature.

### Example 6

1. The same process steps as 1-2 in Example 5.

2. Where the mycological biopolymer is infused with a probiotic or other microorganism, such as a microorganism designed to produce and excrete desired compounds such as anti-inflammatory agents, collagen, silk protein, cannabidiol (CBD) and other skin care ingredients.

### Example 7

1. The same process steps as 1-2 in Example 5.

2. Where the mycological biopolymer is subjected to a post-process (e.g., chopping or grinding) to achieve individual particles.
a. Particle size ranging firm 100 microns to 1 cm in diameter.

3. Where those particles serve as a hydro-bead, a moisturizing agent, and an exfoliant.

4. Where those particles are mixed or infused with other ingredients as detailed in Examples 5 and 6.

A mycological biopolymer is grown such that exogenous pigments are induced during the growth of the biomaterial. Pigment production can be induced using environmental or incubation stimuli, nutritional inducers within the growth media or substrate, through genetic engineering to either upregulate native genes or knock-out those that may inhibit biochemical pathways, or a combination thereof.

### Example 9

1. The same process steps as 1-2 in Example 5.

2. Where the mycological biopolymer is infused with a pigment, to be applied to the skin or lips.

### Example 10

1. The same process steps as 1-2 in Example 1.

2. The extracted tissue is deacetylated to transform the chitinous, skeletal layer of the fungal cell walls in the tissue to chitosan to impart anti-microbial properties.

3. Step “2” is followed by steps 3-7 in Example 1.

### Example 11

1. A mycological biopolymer is grown such that beta-glucan production is induced during the growth of the biomaterial. The mycological biopolymer is in the form of a mat having dimensions of at least 10″ by 6″ by 1″.

2. The mat density may vary from 0.25 pounds per cubic foot to 3 pounds per cubic foot.

3. Mat is extracted and then either
a. Dried and then cut into a series of cosmetics applicators in the shape of triangles, orbs, cubes, and other shapes commonly used to apply makeup; or
b. Die cut into a series of cosmetics applicators in the shape of triangles, orbs, cubes, and other shapes commonly used to apply makeup and then dried.

### Example 1

As in Example 1, but the mycological biopolymer is sliced to between 50 and 500 microns and applied as a film or mask to skin.

The disclosure thus provides an applicator for a health and/or beauty product that is biodegradable and that can be custom made for the application of a health or beauty product.

### Example 13

A mycological biopolymer is grown essentially as disclosed herein. The mycelial mat is removed from the growth substrate and compressed into the desired form or shape using a heated platen press held at a temperature between 200-300° F., a force of 100,000 lbf across the platen, and a hold time no greater than 60 seconds.

We claim:

1. A topical applicator comprising a structural matrix, wherein the structural matrix comprises a mycological biopolymer.

2. The topical applicator of claim 1, wherein the topical applicator is suitable for use in applying a health or beauty product to an application region of a subject.

3. The topical applicator of claim 1 for use in applying a health or beauty product to an application region of a subject.

4. The topical applicator of claim 2 or 3, wherein the application region is the subject's skin or lips.

5. The topical applicator of any one of claims 1 to 4, wherein the mycological biopolymer is produced by a method comprising:
providing a growth medium comprising a fungal inoculum and a substrate, said substrate optionally further comprising a supplemental source of nutrition, and said fungal inoculum comprising a fungus; and
incubating the growth medium as a solid-state culture for a period of time in a growth environment, wherein the growth environment has a relative humidity, a temperature, carbon dioxide (CO₂) and oxygen (O₂) sufficient to support growth of the mycological biopolymer without the formation of a visible fruiting body;
thereby providing the mycological biopolymer.

6. The topical applicator of claim 5, wherein the growth environment carbon dioxide level is within a range of about 3% (v/v) to about 7% (v/v).

7. The topical applicator of claim 6, wherein the growth environment carbon dioxide level is within the range of about 5% (v/v) to about 7% (v/v).

8. The topical applicator of any one of claims 5 to 7, wherein the growth environment relative humidity is at least about 95%.

9. The topical applicator of claim 8, wherein the growth environment relative humidity is about 99%.

10. The topical applicator of any one of claims 5 to 9, wherein the growth environment has a temperature ranging from about 55° F. to about 100° F.

11. The topical applicator of claim 10, wherein the growth environment has a temperature ranging from about 85° F. to about 95° F., or from about 85° F. to about 90° F.

12. The topical applicator of any one of claims 5 to 11, wherein the fungus is a species of a genus selected from the group consisting of Flammulina Ganoderma, Inonotus, Lentinula, Morchella and Trametes.

13. The topical applicator of any one of the preceding claims, wherein the mycological biopolymer has a density ranging from about 0.1 to about 5 pounds per cubic foot.

14. The topical applicator of any one of the preceding claims, wherein the mycological biopolymer has an open volume ranging from about 70% to about 99%, about 75% to about 99%, about 80% to about 99%, or about 80% to about 92% (v/v).

15. The topical applicator of claim 13 or 14, wherein the mycological biopolymer has a density of at least about 3 pounds per cubic foot.

16. The topical applicator of claim 15, wherein the growth environment has a horizontal directed air flow, said air flow having a rate ranging from about 250 feet per minute to about 500 feet per minute, or about 275 feet per minute to about 500 feet per minute.

17. The topical applicator of claim 15 or 16, wherein the mycological biopolymer has a tensile strength ranging from about 25 to about 35 psi.

18. The topical applicator of claim 13 or 14, wherein the mycological biopolymer has a density ranging from about 0.8 to about 3 pounds per cubic foot.

19. The topical applicator of claim 18, wherein the growth environment has a horizontal directed air flow, said air flow having a rate ranging from about 100 feet per minute to about 250 feet per minute, or about 110 to about 250 feet per minute.

20. The topical applicator of claim 18 or 19 wherein the mycological biopolymer has a tensile strength ranging from about 10 to about 20 psi.

21. The topical applicator of claim 13 or 14, wherein the mycological biopolymer has a density ranging from about 0.5 to about 2 pounds per cubic foot.

22. The topical applicator of claim 21, wherein the growth environment has a horizontal directed air flow, said air-flow having an air flow rate of at most about 100 feet per minute, or less than about 100 feet per minute.

23. The topical applicator of any one of claims 12 to 22, wherein the fungus is a species of the genus Ganoderma.

24. The topical applicator of claim 23, wherein the fungus is Ganoderma lucidum.

25. The topical applicator of claim 13 or 14, wherein the mycological biopolymer has a density of less than about 1 pound per cubic foot.

26. The topical applicator of claim 25, wherein the fungus is a species of the genus Morchella.

27. The topical applicator of any one of claims 5 to 26, wherein the growth medium is incubated in the growth environment as the solid-state culture for a period of time of up to about 3 weeks.

28. The topical applicator of claim 27, wherein the period of time is from about 4 days to about 14 days.

29. The topical applicator of claim 28, wherein the period of time is about 9 days.

30. The topical applicator of any one of the preceding claims, wherein the mycological biopolymer has a fluid retention capacity of up to about 20-fold of the dry mass of the mycological biopolymer.

31. The topical applicator of any one of claims 1 to 11, wherein the mycological biopolymer comprises hyphal filaments having a thickness ranging from about 0.5 micron to about 10 microns, or from about 0.5 micron to about 6 microns.

32. The topical applicator of any one of claims 1 to 11, wherein the mycological biopolymer has a modulus of elasticity ranging from about 1 to about 10 psi.

33. The topical applicator of claim 32, wherein the mycological biopolymer has a modulus of elasticity ranging from about 30 to about 60 psi, about 35 to about 60 psi, or about 35 to about 55 psi.

34. The topical applicator of any one of claims 5 to 14, wherein the mycological biopolymer provided by the method has a mat height ranging from about 0.125 inch to about 3 inches.

35. The topical applicator of any one of claims 1 to 11, wherein the mycological biopolymer has a hydrophobicity characterized by a contact angle ranging from about 90 to about 150 degrees, about 100 to about 130 degrees, or about 110 to about 125 degrees.

36. The topical applicator of any one of claims 1 to 11, wherein the mycological biopolymer has a chitin content ranging from about 5% to about 35%, about 5% to about 25%, or about 5% to about 15%, about 8 to about 12%, or about 10% (w/w) of the dry mass of the mycological biopolymer.

37. The topical applicator of any one of claims 1 to 11, wherein the mycological biopolymer has a protein content ranging from about 1% to about 25%, about 2% to about 20%, about 5% to about 10%, or about 6% to about 9% (w/w) of the dry mass of the mycological bipolymer.

38. The topical applicator of any one of the preceding claims, wherein the structural matrix consists of, or consists essentially of the mycological biopolymer.

39. The topical applicator of any one of the preceding claims, wherein the topical applicator consists of the mycological biopolymer.

40. The topical applicator of any one of claims 1 to 38, wherein the topical applicator further comprises an additional substance.

41. The topical applicator of claim 40, wherein the additional substance is a health or beauty product.

42. The topical applicator of claim 41, wherein the additional substance is a health or beauty product suitable for topical administration.

43. The topical applicator of claim 40 or 41, wherein the additional substance is an anesthetic, an analgesic, an antimicrobial, an antibiotic, an antiseptic, an anti-inflammatory, an exfoliant, a cosmetic, a sunscreen, a sun lotion, a moisturizer, a topical burn treatment agent, a cleanser, an astringent, a toner, a chelator, an anti-aging product, an anti-acne agent, an anti-coagulant, a protein, a signaling molecule, a complex carbohydrate, a pigment, a vitamin, a nutritional supplement, an oil or a microbe; or a combination thereof.

44. The topical applicator of claim 43, wherein the additional substance is a protein.

45. The topical applicator of claim 44, wherein the protein is collagen.

46. The topical applicator of claim 43, wherein the additional substance is a complex carbohydrate.

47. The topical applicator of claim 46, wherein the complex carbohydrate is hyaluronic acid, beta-glucan, or a combination thereof.

48. The topical applicator of claim 47, wherein the beta-glucan is water soluble.

49. The topical applicator of claim 40 or 41, wherein the additional substance is added to the mycological biopolymer after the growth of the mycological biopolymer is terminated.

50. The topical applicator of claim 43, wherein the additional substance is a microbe.

51. The topical applicator of claim 50, wherein the microbe is added during the growth of the mycological biopolymer.

52. The topical applicator of any one of the preceding claims, wherein the topical applicator is provided in the form of a sheet, a circular disc, a triangle, a cylinder, a rectangle or a cone, or any other 3-dimensional molded form or custom design.

53. The topical applicator of any one of claims 1 to 51, wherein the topical applicator is a mask, a finger spacer, a toe spacer, a cleansing foam, a wipe, an ear plug, a product applicator or a product blender.

54. The topical applicator of any one of claims 1 to 53 for use in the application or a beauty product.

55. The topical applicator of claim 54, wherein the beauty product is a cosmetic.

56. The topical applicator of any one of claims 1 to 53 for use in the application of a health product.

57. The topical applicator of claim 56, wherein the topical applicator is a bandage, a burn dressing or a wound dressing.

58. The topical applicator of any one of claims 1 to 39, wherein the topical applicator is biodegradable.

59. The topical applicator of any one of claims 5 to 58, wherein the growth medium substrate is a lignocellulosic substrate.

60. The topical applicator of claim 59, wherein the lignocellulosic substrate is an agricultural waste product selected from the group consisting of corn stover, kenaf pith, canola straw and wheat straw.

61. The topical applicator of claim 59, wherein the lignocellulosic substrate is not an agricultural waste product.

62. The topical applicator of claim 61, wherein the lignocellulosic substrate is soy flour or maple wood flour.

63. The topical applicator of any one of claims 5 to 58, wherein the growth medium substrate is a cellulosic substrate.

64. The topical applicator of claim 63, wherein the cellulosic substrate is a lignin-free material.

65. The topical applicator of any one of claims 5 to 58, wherein the growth medium substrate is an inorganic material.

66. The topical applicator of claim 65, wherein the inorganic material is vermiculite, perlite, soils, chalk, gypsum, clay, sand, rockwool, expanded clay or growstones.

67. The topical applicator of claim 5, wherein the fungus is genetically engineered to overexpress a chitin deacetylase (DCA) gene.

68. The topical applicator of claim 5, wherein the fungus is genetically engineered to overexpress hydrophobins.

69. The topical applicator of claim 5, wherein the fungus is of the genus Ganoderma genetically engineered to overexpress the genes BGS1 and BGS2 that encode the two β-1,3-glucan synthases therein.

70. A method of enhancing the health a subject, the method comprising using a topical applicator of any one of claims 1 to 69 to apply a health product to an application region of a subject in need thereof.

71. A method of enhancing the aesthetic appearance of a subject, the method comprising using a topical applicator of any one of claims 1 to 69 to apply a beauty product to an application region of a subject in need thereof.
